(19) United States
(12) Patent Application Publication
(10) Pub. No.: US 2022/0387268 A1
(43) Pub. Date: Dec. 8, 2022

(54) OIL-IN-WATER MACROEMULSION

(71) Applicant: SYMRISE AG, Holzminden (DE)

(72) Inventors: Joern Wiedemann, Holzminden (DE); Joerg Droege, Bodenwerder (DE); Julia Von Holly-Ponientzietz, Holzminden (DE); Matthias Wuensch, Holzminden (DE); Thorsten Boeddeker, Brakel (DE)

(21) Appl. No.: 17/611,891

(22) PCT Filed: May 15, 2020
(86) PCT No.: PCT/EP2020/063736

§ 371 Date: Nov. 16, 2021

(30) Foreign Application Priority Data
May 17, 2019 (EP) PCT/EP2019/062816

(51) Int. Cl. A61K 8/06; A61K 9/107; A01N 25/30; A01N 25/04; A61Q 19/00; C11D 3/50; C11D 17/00; A23K 50/40; A23L 35/00

(52) U.S. Cl. CPC A61K 8/062; A61K 9/107; A01N 25/30; A01N 25/04; A61Q 19/00; C11D 3/50; C11D 17/0017; A23K 50/40; A23L 35/10; A23V 2002/00

(57) ABSTRACT

The present invention relates to the field of liquid solubilising systems. More specifically, the present invention relates to a macroemulsion comprising a hydrophobic active substance, which enhances the transfer of said hydrophobic active substance to a subject or object. A method for producing the macroemulsion, and the use of said macroemulsion to provide a sensory effect or other benefits, are likewise provided. The present invention also pertains to the use of the macroemulsion for preparing consumer products. Consumer products comprising or consisting of said macroemulsion, such as foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, laundry scent, laundry scent boosters, pharmaceuticals and pet foods, in particular laundry and cleaning agents, also form part of the present invention. The present invention especially pertains to laundry scent or laundry scent booster products which impart laundry an intensive fragrance effect.

### FIELD OF THE INVENTION

The present invention relates to the field of liquid solubilising systems. More specifically, the present invention relates to an oil-in-water macroemulsion comprising a hydrophobic active substance, which enhances the transfer of said hydrophobic active substance to a subject or object. A method for producing the oil-in-water macroemulsion, and the use of said macroemulsion to provide a sensory effect or other benefits, are likewise provided. The present invention also pertains to the use of the oil-in-water macroemulsion for preparing consumer products. Consumer products comprising or consisting of said macroemulsion, such as foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, laundry scent, laundry scent boosters, pharmaceuticals and pet foods, in particular laundry and cleaning agents, also form part of the present invention. The present invention especially pertains to laundry scent or laundry scent booster products which impart laundry an intensive fragrance effect.

### BACKGROUND OF THE INVENTION

Microemulsions comprising one or more hydrophobic or lipophilic active substances are known in the prior art. Microemulsions are clear, thermodynamically stable, isotropic liquid mixtures of oil, water and surfactant, frequently in combination with a cosurfactant. Microemulsions are formed upon simple mixing of the components and do not require the high shear conditions generally used in the formation of ordinary emulsions. Their droplets vary in diameter from about 1 to 100 nm, usually 10 to 50 nm.

Microemulsions have many commercially important uses, for example in detergents, foods, cosmetics, pharmaceutics and agrochemicals.

Microemulsions have been proven to increase the absorbance of cosmetic ingredients such as whitening agents, moisturisers and antioxidants.

In pharmacy, microemulsions have potential suitability for dermal and transdermal administration of a wide variety of drug molecules. Due to a more pronounced solubilising property resulting in high concentration gradients and an increased dermal drug permeation rate, microemulsions have better bioavailability than conventional emulsions.

In laundry products such as liquid detergents, fabric softeners, laundry scent or laundry perfumes, scent boosters or cleaning agents, hydrophobic or lipophilic active substances such as perfumes are usually transferred to fabrics during the laundry process or to a surface by microemulsions with the active substances solubilised by larger amounts of surfactant. In personal care applications such as hair conditioners, formulation chemists approach the transfer of active substances to hair, beard or fur in the same way.

However, preparing microemulsions requires a considerable concentration of surfactant. Additionally, microemulsions remain stable when diluted in water, for example in a laundry or washing or cleaning process, and their main part is deposited in the water and flushed away during washing. Consequently, in the transfer of for example perfume oils from liquid detergents, fabric softeners, laundry scent or laundry perfumes, etc., very high excesses of active substances have to be provided in order to compensate for this wastage.

Additionally, in the microemulsion approach there is often a problem with generating a stable suspension of microcapsules comprising a hydrophobic or lipophilic perfume substance, as is the case in the laundry or cleaning sectors. Due to the low Newtonian viscosity of the formulation, microcapsules would sediment.

Another common practice in the state of the art for perfume oil transfer involves fragranced powders or pastilles and clear-to-opaque liquids. The perfume substances or perfume oil is/are directly mixed into and solubilised in the product, for example a fabric softener, detergent or the like. The perfume substances or perfume oils can be delivered to the fabric through the rinse cycle of a washing machine.

There are, however, several drawbacks to these simple approaches. Many fragrances substances are volatile, resulting in fragrance loss during manufacturing, storage and use. Many fragrance oils are also unstable over time. Another problem in the art is that in the transfer of for example perfume substances or perfume oils from said products, most of their active substances are washed away during washing, i.e. the carryover of active substances is rather marginal, i.e. much active substance such as fragrance is lost during washing and is not deposited on the fabrics and end up in sewage. Very high excesses of active compounds have to be provided in order to compensate for this inefficient delivery, causing increased expense and pollution of sewage water.

As consumers continue to demand fragranced products, the perfuming of consumer products is an increasingly significant challenge.

Various methods for solving these problems have been employed in the art. One approach is to encapsulate the fragrance oils. However, encapsulation can be costly. Another approach is to use solid carriers for perfume oils. Carrier materials can be organic or inorganic in nature, such as for example starches, silicic acids, phosphates, zeolites, alkali salts of polycarboxylic acids, cyclodextrins, etc. One of the problems with using carriers is that the loading or concentration of the perfume oil can be limited, thereby imparting insufficient or suboptimal amounts of perfume oil to the laundry product in a stable form that will be available for fabric deposition.

EP 1 297 101 A2 discloses a laundry composition with reduced surfactant concentration.

EP 1 265 645 A1 discloses a perfume composition in which perfume oil is dispersed in a solid matrix based on a molten polymer mass.

WO 2018/073 238 A1 pertains to a ringing gel composition i.e. a self-thickened composition having a viscoelastic behaviour and a viscosity of between 0.1 and 1000 Pas at 20° C., said ringing gel comprising an aqueous phase, a surfactant system essentially consisting of non-ionic surfactant(s), a linker and an oil phase comprising a hydrophobic active ingredient.

US 2017/226690 A1 relates to mixtures of perfume oil and capsules which are stabilised by large concentrations of polyethylene glycol.

Accordingly, there is an ongoing need to improve fragrance (oil) loading capability, fragrance (oil) stability, fragrance (oil) transfer, i.e. fragrance delivery to, or fragrance (oil) deposition on subjects and objects, in particular on fabrics, and to achieve the fragrance effect for a longer period of time.

It is therefore an object of the present invention to provide formulations, and products made from the same, for an improved provision, transfer and deposition of an effective amount of a selected hydrophobic active substance.

It is also an object of the present invention to provide formulations, and products made from the same, for an improved provision, transfer and deposition of an effective amount of a selected perfume to a laundry wash and/or rinse solution.

It is yet another object of the present invention to provide a method for preparing such formulations.

It is yet another object of the present invention to provide a method for an improved provision, transfer and deposition of an effective amount of a selected hydrophobic active substance.

These and other objects and advantages of the present invention will become clear from the following disclosure.

Surprisingly, it has now been found that a macroemulsion with a reduced surfactant concentration and a considerably larger droplet size allows much higher loading, effective transfer, i.e. carryover, and deposition of hydrophobic or lipophilic active substances than with common microemulsions. The resulting enhancement of the transfer of hydrophobic or lipophilic active substances is remarkable.

### SUMMARY OF THE INVENTION

In a first aspect, the present invention relates to an oil-in-water macroemulsion comprising or consisting of:
(a) an aqueous phase;
(b) an oily phase comprising at least one hydrophobic active substance, in particular a perfume or aroma substance;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof; and optionally
(e) at least one other additive and/or adjuvant;
   wherein the amount of the surfactant is from 0.4 to 25% by weight, based on the total weight of the oily phase.

In a second aspect, the present invention relates to a method for preparing an oil-in-water macroemulsion, comprising the steps of:
(1-i) providing an aqueous phase by mixing and dissolving at least one stabiliser, and optionally at least one other additive and/or adjuvant, in an aqueous solution;
(1-ii) providing an oily phase by mixing and dissolving at least one hydrophobic active substance and/or optionally at least one hydrophobic active substance in a microcapsule form, and at least one surfactant in an oily solution; and
(1-iii) dispersing the oily phase in the aqueous phase by stirring, shaking, pressing or otherwise inducing sheer forces, to obtain a macroemulsion;
   or
(2-i) providing an aqueous phase by mixing and dissolving at least one surfactant, at least one stabiliser and optionally at least one other additive and/or adjuvant in an aqueous solution;
(2-ii) providing an oily phase by mixing and dissolving at least one hydrophobic active substance and/or optionally at least one hydrophobic active substance in a microcapsule form in an oily solution; and
(2-iii) dispersing the oily phase in the aqueous phase by stirring, shaking, pressing or otherwise inducing sheer forces, to obtain a macroemulsion;
   wherein the amount of the surfactant is from 0.4 to 25% by weight, based on the total weight of the oily phase.

In a third aspect, the present invention relates to a method for providing, transferring and depositing a hydrophobic active substance, comprising the steps of:
providing an oil-in-water macroemulsion according to the present invention; and
bringing the oil-in-water macroemulsion, in particular a perfume or an aroma macroemulsion, in contact with a subject or object.

In a forth aspect, the present invention relates to the use of a macroemulsion according to the present invention in a perfume, flavouring, active skin-product ingredients, active pharmaceutical ingredients, dyes, UV-active substances, optical brighteners, bodying agents, drape and form control agents, smoothness agents, static control agents, wrinkle control agents, sanitising agents, disinfecting agents, germ control agents, mould control agents, mildew control agents, antiviral agents, antimicrobials, drying agents, stain resistance agents, soil release agents, malodour control agents, fabric freshening agents, dye fixatives, colour maintenance agents, colour restoring/rejuvenating agents, anti-fading agents, anti-abrasion agents, wear resistance agents, fabric integrity agents, anti-wear agents, rinsing aids, UV protection agents, sun fade inhibitors, insect repellents, anti-allergenic agents, flame retardants, water-proofing agents, fabric softening agents, shrinkage resistance agents or stretch resistance agents.

In a fifth aspect, the present invention relates to the use of an oil-in-water macroemulsion according to the present invention as an ingredient for preparing foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, laundry scent, laundry scent boosters, pharmaceuticals and pet foods, in particular laundry scent and laundry scent products.

Finally, a sixth aspect of the present invention relates to foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, laundry scent, laundry scent boosters, pharmaceuticals and pet foods comprising a macroemulsion according to the present invention, in particular laundry scent and laundry scent products.

### FIGURES

FIG. 1 shows the influence of the stirring speed on the droplet size of the oil-in-water emulsion.

FIG. 2 shows the variation in the surfactants of the oil-in-water emulsion.

FIG. 3 shows the influence of the Genaminox® concentration on the droplet size of the emulsion.

FIG. 4 shows a compilation of the most favourable surfactant compositions and their corresponding ratios.

FIG. 5 shows the influence of the Tween 20 concentration on the droplet size of the emulsion.

FIG. 6 shows the influence of the surfactant concentration on the droplet size of the emulsion.

FIG. 7 shows the increase in the droplet size subject to the surfactant concentration and, simultaneously, the stirring speed.

FIGS. 8 and 9 show the results of a sensory evaluation of the emulsion according to the present invention.

FIG. 10 shows the quantitative determination of perfume oil adsorption on cloth via SDE and GC.

FIG. 11 shows the total amount of perfume oil adhering to a piece of towel after a washing procedure using a scent lotion according to the present invention.

FIG. 12 shows the elimination of germs by an oil-in-water macroemulsion according to the present invention.

FIG. 13 shows the droplet size distribution of the oil-in-water macroemulsion according to the present invention with a non-ionic solubilizer at different sheer stress.

FIG. 14 shows the droplet size distribution of the oil-in-water macroemulsion according to the present invention with an anionic surfactant mixture at different sheer stress.

FIG. 15 shows the droplet size distribution of the oil-in-water macroemulsion according to the present invention with another anionic surfactant mixture at different sheer stress.

FIG. 16 shows the droplet size distribution of the oil-in-water macroemulsion according to the present invention with a cationic surfactant mixture at different sheer stress.

FIG. 17 shows the droplet size distribution of the oil-in-water macroemulsion according to the present invention with another cationic surfactant mixture at different sheer stress.

FIG. 18 represents an assembly of scent lotion formulations prepared from different surfactant compositions with similar droplet sizes adjusted by the applied sheer stress.

FIG. 19 shows the quantification of perfume oil transfer to the fabric of the scent lotion formulations shown in FIG. 18.

FIG. 20 represents samples of the scent lotion formulations prepared from different surfactant compositions at different sheer stress and their instability index.

FIG. 21 is a visualization of the correlation between droplet size and instability index.

FIG. 22 represents a combination of elasticity and loss modulus: tan(d)=G′/G″ in dependence of the temperature of five different samples of scent lotion formulations prepared from different surfactant compositions.

FIG. 23 represents a combination of elasticity and loss modulus: tan(d)=G′/G″ in dependence of the temperature of five different samples of scent lotion formulations prepared from different surfactant compositions.

FIG. 24 shows the relation between droplet size and perfume concentration.

FIGS. 25a and 25b show the relative quantitative transfer of a fragrance compound in the washing/laundry process to cloth in dependence on the droplet size, the water solubility and log P value of the fragrance compound.

FIG. 26 is a table showing a comparison of the stability of scent lotions prepared from different thickeners.

### DETAILED DESCRIPTION

The present invention relates to an oil-in-water macroemulsion prepared from a hydrophobic active substance, water, a surfactant and a stabiliser for stabilising the macroemulsion.

Macroemulsions are mixtures of two immiscible liquids, one of them being dispersed in the other liquid in the form of fine droplets with a diameter of greater than 0.1 μm. Such systems are homogenous, transparent, milky in colour and thermodynamically unstable, i.e. the macroemulsion will ultimately separate into the two original immiscible liquids over time. They are part of a larger family of emulsions along with microemulsions. As with all emulsions, one phase serves as the dispersing agent. This is often called the continuous or outer phase. The remaining phase is called the dispersed or inner phase, because the liquid droplets are finely distributed amongst the larger continuous-phase droplets. This type of emulsion is thermodynamically unstable, but can be stabilised for a period of time with the application of kinetic energy. Emulsifiers (surfactants) are used to reduce the interfacial tension between the two layers and so induce macroemulsion stability for a useful amount of time.

Single macroemulsions, comprising two phases, can be subdivided into two different types. In an oil-in-water (O/W) emulsion, oil droplets are dispersed in water. This is the most common type of emulsion. Conversely, a water-in-oil (W/O) emulsion involves water droplets finely dispersed in oil.

Within the context of the present invention, the macroemulsion is an oil-in-water (O/W) emulsion in which the oily phase is dispersed in an aqueous phase.

The first component, constituting the aqueous or continuous or outer phase of the oil-in-water macroemulsion of the present invention, is water or alternatively water in combination with a polar solvent, miscible in water but immiscible in the oily phase. Specific non-limiting examples of such water-miscible solvents include ethanol, DMSO (dimethyl sulfoxide), DPG (dipropylene glycol) or PEG (polyethylene glycol.

Preferably, the aqueous phase of the oil-in-water macroemulsion of the present invention is pure water.

The proportion of the aqueous or continuous phase is from 25 to 99.9% by weight, preferably from 50 to 99 by weight, more preferred from 80 to 98% by weight, particularly preferred from 90 to 97% by weight, and most preferred from 93 to 96% by weight, relative to the total weight of the oil-in-water macroemulsion composition.

The second component, constituting the oily phase or dispersed or inner phase of the oil-in-water macroemulsion of the present invention, is the hydrophobic active substance itself or alternatively the hydrophobic active substance in mixture with a non-polar solvent immiscible with water. Specific non-limiting examples of such solvents include for example animal or vegetable oils or fats or their hydrolysates, paraffin oils, silicones, isopropyl myristate, DPG (dipropylene glycol), DPM (Dipropylene glycol methyl ether), ethanol, isopropanol, glycol, glycerol derivatives, triethylcitrate, triacetin, benzyl benzoate, MMB (3-Methoxy-3-methyl-1-butanol), Isopar L® (C11-13 Isoparaffin), neononyl acetate, dioctyl adipate, propylene carbonate, ethyl acetoacetate.

In a preferred variant, the oily phase of the oil-in-water macroemulsion of the present invention is solely the hydrophobic active substance.

The proportion of the oily or dispersed phase is 0.1 to 75% by weight, preferably 1 to 50% by weight, more preferred 2 to 20% by weight, particularly preferred 3 to 10% by weight, and most preferred 4 to 7% by weight, relative to the total weight of the oil-in-water macroemulsion composition.

The mixing ratio of the aqueous or continuous phase to the oily or dispersed phase is preferably within a range of 999.09:0.01 to 250:750, preferably 990:10 to 500:500, more preferably 900:100 to 800:200.

The oily or dispersed phase of the oil-in-water macroemulsion according to the present invention comprises at least one hydrophobic active substance.

The term “active substance”, as used in the present invention, is to be understood to mean a substance or ingredient pertaining to a certain effect, for example a fragrance, an aroma, a dye, a pharmaceutical drug, a pesticide, etc.

The active substances in the macroemulsion of the present invention are hydrophobic or lipophilic substances. Hydrophobic substances (hydrophobes) tend to be non-polar and therefore prefer other neutral molecules and non-polar solvents. Because water molecules are polar, hydrophobic active substances do not dissolve well in water, but rather in the oily phase of the macroemulsion.

In the macroemulsion of the present invention, the hydrophobic substance is either part of the oily or inner phase, i.e. with another component constituting the oily or dispersed phase of the macroemulsion, or itself constitutes the oily or dispersed phase of the macroemulsion.

Preferably, the hydrophobic substance alone constitutes the oily or dispersed phase of the macroemulsion.

In accordance with the present invention, the at least one hydrophobic active substance is preferably selected from the group consisting of perfume substances, perfume oils, aroma substances, aromas, active skin-product ingredients, active pharmaceutical ingredients, dyes, UV-active substances, optical brighteners, bodying agents, drape and form control agents, smoothness agents, static control agents, wrinkle control agents, sanitising agents, disinfecting agents, germ control agents, mould control agents, mildew control agents, antiviral agents, antimicrobials, drying agents, stain resistance agents, soil release agents, malodour control agents, fabric freshening agents, dye fixatives, colour maintenance agents, colour restoring/rejuvenating agents, anti-fading agents, anti-abrasion agents, wear resistance agents, fabric integrity agents, anti-wear agents, rinsing aids, UV protection agents, sun fade inhibitors, insect repellents, anti-allergenic agents, flame retardants, water-proofing agents, fabric softening agents, shrinkage resistance agents and stretch resistance agents.

The hydrophobic active substances and other ingredients present in the oil-in-water macroemulsion according to the invention are described in detail hereinafter. However, the lists of these active substances and other ingredients are non-limiting and may include further active ingredients and other ingredients that are not elucidated further below.

In order to improve the esthetic impression of the textile treatment agents, they can be colored using suitable dyes. Preferred dyes that are easy for the person having ordinary skill in the art to select have high storage stability, are insensitive to light, and do not show pronounced substantivity with respect to textile fibers so that they will not stain the latter.

As dyes, dye substances can be used that are for example listed in “Kosmetische Färbemittel” der Farbstoffkommission der Deutschen Forschungsgemeinschaft, Verlag Chemie, Weinheit, 1984, pages 81-106. Examples are Cochenille Red A (C.I. 16255), Patent blue V (C.I. 42051), Indigotin (C.I. 73015), Chlorophyllin (C.I. 75810), Quinolin Yellow (C.I. 47005); Titandioxid (C.I. 77891), Indanthren Blue RS (C.I. 69800) and Krapp Lacquer (C.I. 58000). As luminescent dye, also Luminol may be comprised.

Suitable soil release polymers, which are also referred to as “antiredeposition agents”, are for example non-ionic cellulose ethers such as methyl cellulose and methylhydroxypropyl cellulose having a proportion of methoxy groups of 15% to 30% by weight and of hydroxypropyl groups of 1% to 15% by weight, based in each case on the non-ionic cellulose ethers, and also the polymers, known from the prior art, of phthalic acid and/or terephthalic acid or of derivatives thereof, especially polymers of ethylene terephthalates and/or polyethylene and/or polypropylene glycol terephthalates or anionically and/or non-ionically modified derivatives of these. Suitable derivatives include the sulphonated derivatives of phthalic acid and terephthalic acid polymers.

Optical brighteners (so-called “whitening agents”) can be added to the macroemulsion in order to eliminate greying and yellowing of the treated textile surfaces. These substances are absorbed by the fibers, brightening them and simulating a bleaching effect, by converting invisible ultraviolet radiation into visible longer-wavelength light, wherein the ultraviolet light absorbed from sunlight is given off as a slightly blue fluorescence, combining with the yellow color of the greyed or yellowed laundry to give a pure white color. Suitable compounds are derived for example from the substance classes of the 4,4′-diamino-2,2′-stilbene disulfonic acids (flavonic acids), 4,4′-distyryl-biphenylenes, methyl umbelliferones, coumarins, dihydroquinolinones, 1,3-diarylpyrazolines, naphthalic acid imides, benzoxazole, benzisoxazole and benzimidazole systems, and heterocycle-substituted pyrene derivatives.

Greying inhibitors have the task of keeping the dirt detached from the fibres suspended in the liquor and hence of preventing reattachment of the dirt. Suitable for this purpose are water-soluble colloids, usually organic in nature, for example size, gelatin, salts of ethersulphonic acids of starch or of cellulose or salts of acidic sulphuric acid esters of cellulose or of starch. Also suitable for this purpose are water-soluble polyamides containing acidic groups. In addition, it is possible to use soluble starch preparations and starch products other than those specified above, for example degraded starch, aldehyde starches etc. It is also possible to use polyvinylpyrrolidone. However, preference is given to using cellulose ethers such as carboxymethyl cellulose (sodium salt), methyl cellulose, hydroxyalkyl cellulose and mixed ethers such as methylhydroxyethyl cellulose, methylhydroxypropyl cellulose, methylcarboxymethyl cellulose and mixtures thereof.

Since textile fabrics, especially made of rayon, cellulose, cotton and mixtures thereof, can have a tendency to crease because the individual fibres are sensitive to bending, folding, pressing and squashing transverse to the fibre direction, the oil-in-water macroemulsion may contain synthetic anticrease agents. These include, for example, synthetic products based on fatty acids, fatty acid esters, fatty acid amides, fatty acid alkylol esters, fatty acid alkylolamides or fatty alcohols, which have usually been reacted with ethylene oxide, or products based on lecithin or modified phosphoric acid esters.

Increased wearing comfort can be achieved by the additional use of antistatics. Antistatics increase the surface conductivity, thus allowing built-up charges to be more easily discharged. As a rule, antistatics are substances with at least one hydrophilic molecular ligand that provide a more or less hygroscopic film on surfaces. These antistatics, which are usually surface-active, can be divided into nitrogen-containing (amines, amides, quaternary ammonium compounds), phosphorus-containing (phosphoric acid esters) and sulfur-containing (alkyl sulfonates, alkyl sulfates) antistatics. Lauryl-(or stearyl)dimethylbenzyl ammonium chlorides are suitable as antistatics for textile surfaces or as additives to detergents and cleaning agents, wherein an additional conditioning effect is also achieved.

Humectants serve to regulate moisture on the skin. Humectants preferred according to the invention include amino acids, pyrrolidone carboxylic acid, lactic acid and salts thereof, lactitol, urea and urea derivatives, uric acid, glucosamine, creatinine, cleavage products of collagen, chitosan or chitosan salts/derivatives, and in particular polyols and polyol derivatives (e.g. glycerol, diglycerol, triglycerol, ethylene glycol, propylene glycol, butylene glycol, erythritol, 1,2,6-hexane triol, polyethylene glycols such as PEG-4, PEG-6, PEG-7, PEG-8, PEG-9, PEG-10, PEG-12, PEG-14, PEG-16, PEG-18, PEG-20), sugar and sugar derivatives (including fructose, glucose, maltose, maltitol, mannitol, inositol, sorbitol, sorbitol silane diol, sucrose, trehalose, xylose, xylitol, glucuronic acid and salts thereof), ethoxylated sorbitol (Sorbeth-6, Sorbeth-20, Sorbeth-30, Sorbeth-40), honey and hardened honey, hardened starch hydrolysates and mixtures of hardened wheat protein and PEG-20-acetate copolymer. Preferred according to the invention as suitable humectants are glycerol, diglycerol, triglycerol and butylene glycol.

Biogenic agents are understood for example to refer to tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts such as e.g. prune extract, Bambara nut extract and vitamin complexes.

In order to prevent undesired changes in the treated textile surfaces caused by the action of oxygen and other oxidative processes, the macroemulsion can comprise antioxidants. Examples of compounds of this class include amino acids (e.g. glycine, histidine, tyrosine, tryptophan) and derivatives thereof, imidazoles (e.g. urocanic acid) and derivatives thereof, peptides such as D,L carnosine, D carnosine, L carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cysteamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfone, penta-, hexa-, heptathionine sulfoximine) in very low tolerable dosages (e.g. pmol to μmol/kg), further (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin-A-palmitate) and coniferyl benzoate from benzoic resin, rutinic acid and derivatives thereof, α-glycosylrutin, ferulic acid, furfurylidene glucitol, carnosine, butylhydroxytoluene, butylhydroxyanisole, nordihydroguaiaretic acid, trihydroxybutyrophenone, uric acid and derivatives thereof, mannose and derivatives thereof, superoxide dismutase, zinc and derivatives thereof (e.g. ZnO, ZnSO4) selenium and derivatives thereof (e.g. selenium methionine), stilbenes and derivatives thereof (e.g. stilbene oxide, trans-stilbene oxide) and suitable derivatives according to the invention of the above-mentioned agents (salts, esters, ethers, sugars, nucleotides, nucleosides, peptides and lipids).

Suitable anti-dandruff agents are piroctone olamine (1-hydroxy-4-methyl-6-(2,4,4-trimythylpentyl)-2-(1H)-pyridinone monoethanolamine salt), Baypival® (climbazole), Ketoconazol®, (4-acetyl-1-{-4-[2-(2,4-dichlorphenyl) r-2-(1H-imidazol-1-ylmethyl)-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur-tar distillates, salicylic acid (or in combination with hexachlorophene), undecylenic acid monoethanolamide sulfosuccinate Na-salt, Lamepon® UD (protein-undecylenic acid condensate), zinc pyrithione, aluminum pyrithione and magnesium pyrithione/dipyrithione-magnesium sulfate.

Cosmetic deodorants (deodorizing agents) counteract body odors or mask or eliminate them. Body odors occur due to the effect of skin bacteria on apocrine perspiration, wherein unpleasant-smelling decomposition products are formed. Accordingly, deodorants comprise agents that function as antimicrobial agents, enzyme inhibitors, odor absorbers, and odor-asking agents.

As antimicrobial agents, all substances active against grampositive bacteria are generally suitable, such as e.g. 4-hydroxybenzoic acid and salts and esters thereof, N-(4-chlorophenyl)-N′(3,4-dichlorophenyl)urea, 2,4,4′-trichloro-2′-hydroxy-di-phenylether (triclosan), 4-chloro-3,5-dimethylphenol, 2,2′-methylene-bis(6-bromo-4-chlorophenol), 3-methyl-4-(1-methylethyl)-phenol, 2-benzyl-4-chlorophenol, 3-(4-chlorophenoxy)-1,2-propanediol, 3-iodo-2-propinylbutylcarbamate, chlorohexidine, 3,4,4′-trichlorocarbanilide (TTC), antibacterial fragrances, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides such as e.g. salicylic acid-n-octylamide or salicylic acid-n-decylamide.

Examples of suitable enzyme inhibitors are esterase inhibitors. These are preferably trialkyl citrates such as trimethyl citrate, tripropyl citrate, triisopropyl citrate, tributyl citrate and in particular triethyl citrate (Hydagen® CAT). These substances inhibit enzyme activity and thus reduce the formation of odors. Further substances suitable as esterase inhibitors are sterol sulfates or phosphates, such as e.g. lanosterol, cholesterol, campesterol, stigmasterol and sitosterol sulfates or phosphates, dicarboxylic acids and esters thereof, such as e.g. glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof such as e.g. citric acid, malic acid, tartaric acid or tartaric acid diethyl ester, and zinc glycinate.

Suitable as odor absorbers are substances that are capable of absorbing odor-causing compounds and largely retaining them. They reduce the partial pressure of the individual components and thus reduce their rate of diffusion. It is important in this case that perfumes must remain unaffected. Odor absorbers have no effect against bacteria. They comprise for example as the main component a complex zinc salt of ricinoleic acid or special, largely odor-neutral fragrances that are known to the person having ordinary skill in the art as “fixators,” such as e.g. extracts of labdanum or styrax or certain abietic acid derivatives. Fragrances or perfume oils that, in addition to their function as odor-masking agents, provide deodorants with their respective scents act as odor-masking agents. Examples of perfume oils that can be mentioned are mixtures of natural and synthetic fragrances. Natural fragrances are extracts of flowers, stems and leaves, fruits, fruit peels, roots, woods, hers and grasses, needles and branches, and resins and balsams. Also suitable are animal raw materials such as e.g. civet and castoreum. Typical synthetic fragrance compounds are products of the ester, ether, aldehyde, ketone, alcohol and hydrocarbon types. Fragrance compounds of the ester type are e.g. benzyl acetate, p-tert-butylcyclohexyl acetate, linalyl acetate, phenethyl acetate, linalyl benzoate, benzyl formiate, allylcyclohexyl propionate, styrallyl propionate and benzyl salicylate. Examples of ethers include benzyl ethyl ether, and examples of aldehydes include linear alkanals with 8 to 18 carbon atoms, citral, citronellal, citronellyl oxyacetaldehyde, cyclamen aldehyde, hydroxycitronellal, lilial and bourgeonal, examples of ketones include the ionones and methyl cedryl ketone, alcohols include anethol, citronellol, eugenol, isoeugenol, geraniol, linalool, phenethyl alcohol and terpineol, and hydrocarbons include mainly the terpenes and balsams. Preferred, however, are mixtures of various fragrances that together produce a pleasant scent. Essential oils of low volatility, which are usually used as flavoring components, are also suitable as perfume oils, e.g. sage oil, chamomile oil, clove oil, Melissa oil, mint oil, cinnamon leaf oil, linden flower oil, juniper berry oil, vetiver oil, olibanum oil, galbanum oil, labdanum oil and lavandin oil. Preferred are bergamot oil, dihydromyrcenol, lilial, lyral, citronellol, phenethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-Damascone, geranium oil bourbon, cyclohexyl salicylate, Vertofix Coeur, iso-E-super, Fixolide NP, evernyl, iraldein gamma, phenylacetic acid, geranyl acetate, benzyl acetate, rose oxide, Romilat, Irotyl and Floramat are used alone or in mixtures.

Antiperspirants (antiperspirant agents) reduce the formation of perspiration by affecting the activity of the eccrine sweat glands, thus counteracting underarm wetness and body odor. Aqueous or anhydrous formulations of antiperspirants typically comprise the following ingredients: astringent agents, oil components, nonionic emulsifiers, coemulsifiers, bodying agents, excipients such as e.g. thickeners or complexing agents and/or non-aqueous solvents such as e.g. ethanol, propylene glycol and/or glycerol. Suitable as astringent antiperspirant agents are primarily salts of aluminum, zirconium or zinc. Examples of such suitable antihydrotically active agents are e.g. aluminum chloride, aluminum chlorohydrate, aluminum dichlorohydrate, aluminum sesquichlorohydrate and complex compounds thereof, e.g. with propylene glycol-1,2-aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g. with amino acids such as glycine. In addition, antiperspirants may comprise common oil-soluble and water-soluble excipients in small amounts. Such oil-soluble excipients can for example include: anti-inflammatory, skin-protective, or fragrant essential oils, synthetic skin-protective agents and/or oil-soluble perfume oils.

The macroemulsion can comprise antimicrobial agents in order to combat microorganisms. In this case, a distinction is made depending on the antimicrobial spectrum and mechanism of action between bacteriostates and bactericides, fungistates and fungicides, etc. Examples of important substances from these groups are benzalkonium chlorides, alkylaryl sulfonates, halophenols and phenylmercuric acetates, wherein these compounds may also be dispensed with entirely in the detergents and cleaning agents according to the invention. As antimicrobial agents, all substances active against grampositive bacteria are generally suitable, such as e.g. 4-hydroxybenzoic acid and salts and esters thereof, N-(4-chlorophenyl)-N′(3,4-dichlorophenyl)urea, 2,4,4′-trichloro-2′-hydroxy-di-phenylether (triclosan), 4-chloro-3,5-dimethylphenol, 2,2′-methylene-bis(6-bromo-4-chlorophenol), 3-methyl-4-(1-methylethyl)-phenol, 2-benzyl-4-chlorophenol, 3-(4-chlorophenoxy)-1,2-propanediol, 3-iodo-2-propinylbutylcarbamate, chlorohexidine, 3,4,4′-trichlorocarbanilide (TTC), antibacterial fragrances, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides such as e.g. salicylic acid-n-octylamide or salicylic acid-n-decylamide.

Among the compounds serving as bleaching agents that yield H2O2 in water, sodium perborate tetrahydrate and sodium perborate monohydrate are of particular importance. Further useful bleaching agents are for example sodium percarbonate, peroxypyrophosphates, citrate perhydrates and H2O2-yielding peracidic salts or peracids such as perbenzoates, peroxophthalates, diperazelaic acid, phthaloiminoperacid or diperdodecanedioc acid. Compounds that yield aliphatic peroxocarboxylic acids, preferably with 1 to 10 C atoms, in particular 2 to 4 C atoms, and/or optionally substituted perbenzoic acid under perhydrolysis conditions can be used as bleach activators. Suitable are substances bearing O- and/or N-acyl groups with the above-mentioned number of C atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoyl succinimide (NOSI), acylated phenol sulfonates, in particular N-nonanoyl or isononanoyl oxybenzene sulfonate (n- or iso-NOBS), carboxylic anhydrides, in particular phthalic acid anhydride, and acylated polyhydric alcohols, in particular triacetin, ethylene glycol diacetate and 2,5-diacetoxy-2,5-dihydrofuran. In addition to or instead of the conventional bleach activators, so-called bleach catalysts can also be incorporated into the textile treatment agents. These substances are bleach-enhancing transition metal salts or transition metal complexes such as e.g. Mn-, Fe-, Co-, R- or Mo-salt complexes or -carbonyl complexes. Mn-, Fe-, Co-, Ru-, Mo-, Ti-, V- and Cu-complexes with nitrogen-containing tripod ligands and Co-, Fe-, Cu- and Ru-ammine complexes can also be used as bleach catalysts.

The macroemulsion according to the invention can comprise preservatives. Examples are sorbic acid and salts thereof, benzoic acid and salts thereof, salicylic acid and salts thereof, phenoxyethanol, 3-iodo-2-propynyl butylcarbamate, sodium N-(hydroxymethyl)glycinate, biphenyl-2-ol and mixtures thereof. A suitable preservative is the solvent-free, aqueous combination of diazolidinyl urea, sodium benzoate and potassium sorbate (obtainable as Euxyl® K 500 from Schülke and Mayr), which may be used in a pH range of up to 7. Preservatives based on organic acids and/or salts thereof are particularly suitable for preserving the skin-friendly detergents and oil-in-water macroemulsion according to the invention.

Silicone derivatives, for example, may be used in textile treatment in order to improve rewettability and facilitate ironing of treated textile surfaces. These further improve the rinsing behavior of the detergents and cleaning agents by means of their foam-inhibiting properties. Preferred silicone derivatives are for example polydialkyl or alkylaryl siloxanes in which the alkyl groups have one to five C atoms and are completely or partially fluorinated. Preferred silicones are polydimethylsiloxanes, which can optionally be derivatized and are then aminofunctional or quaternized or have Si—OH, Si—H and/or Si—Cl bonds. The viscosities of the preferred silicones at 25° C. are in the range of 100 to 100,000 m·Pas.

The macroemulsion according to the present invention can also comprise insect repellents. Suitable insect repellents are N,N-diethyl-m-toluamide, 1,2-pentanediol or ethyl butyl acetyl aminopropionate.

The macroemulsion according to the present invention can also comprise UV absorbers that are absorbed onto the treated textile surface and improve the light stability of the fibers. The term UV light protection factors is understood for example to refer to organic substances that are liquid or crystalline at room temperature (light protection filters), which are capable of absorbing ultraviolet rays and giving off the absorbed energy in the form of longer-wavelength radiation, e.g. heat. The UV light protection factors are ordinarily present in amounts of 0.1 to 5 and preferably 0.2 to 1 wt. %. UVB filters can be oil-soluble or water-soluble. Examples of oil-soluble substances include, for example: 3-benzylidene camphor or 3-benzylidene norcamphor and derivatives thereof, e.g. 3-(4-methylbenzylidene) camphor; 4-aminobenzoic acid derivatives, preferably 4-(dimethylamino)benzoic acid-2-ethyl-hexyl ester, 4-(dimethylamino)benzoic acid-2-octyl ester and 4-(dimethylamino)benzoic acid amyl ester; esters of cinnamic acid, preferably 4-methoxycinnamic acid-2-ethylhexyl ester, 4-methoxy-cinnamic acid propyl ester, 4-methoxycinnamic acid isoamyl ester, and 2-cyano-3,3-phenylcinnamic acid-2-ethylhexyl ester(octocrylene); esters of salicylic acid, preferably salicylic acid-2-ethylhexyl ester, salicylic acid-4-iso-propylbenzyl ester, and salicylic acid homomenthyl ester; derivatives of benzophenone, preferably 2-hydroxy-4-methoxybenzophenone, 2-hydroxy-4-methoxy-4′-methylbenzophenone, and 2,2′-dihydroxy-4-methoxybenzophenone; esters of benzyl malonic acid, preferably 4-methoxybenzyl malonic acid di-2-ethylhexyl-ester; triazine derivatives, such as e.g. 2,4,6-trianilino-(p-carbo-2′-ethyl-1′-hexyloxy)-1,3,5-triazine and octyl triazone or dioctyl butamidotriazone (Uvasorb® HEB); propane-1,3-diones, such as e.g. 1-(4-tert-butylphenyl)-3-(4′methoxyphenyl)propane-1,3-dione; ketotricyclo(5.2.1.0)decane derivatives.

In particular, suitable UVA filters include derivatives of benzoyl methane, such as e.g. 1-(4′-tert-butylphenyl)-3-(4′-methoxyphenyl)propane-1,3-dione, 4-tert-butyl-4′-methoxydibenzoyl methane (Parsol® 1789), 2-(4-diethylamino-2-hydroxybenzoyl)-benzoic acid hexyl ester (Uvinul® A Plus), 1-phenyl-3-(4′-isopropylphenyl)-propane-1,3-dione and enamine compounds. The UVA and UVB filters can of course also be used in mixtures. Particularly favorable combinations are composed of the derivatives of benzoylmethane, e.g. 4-tert-butyl-4′-methoxydibenzoylmethane (Parsol® 1789) and 2-cyano-3,3-phenylcinnamic acid-2-ethyl-hexyl ester (octocrylene) in combination with esters of cinnamic acid, preferably 4-methoxycinnamic acid-2-ethylhexyl ester and/or 4-methoxycinnamic acid propyl ester and/or 4-methoxycinnamic acid isoamyl ester. Advantageously, these combinations are used together with water-soluble filters such as e.g. 2-phenylbenzimidazole-5-sulfonic acid and alkali, alkaline earth, ammonium, alkylammoium, alkanolammonium- and glucammonium salts thereof.

In addition to the above-mentioned soluble substances, insoluble light protection pigments, specifically finely-dispersed metal oxides or salts, are also suitable for this purpose. Examples of suitable metal oxides are in particular zinc oxide and titanium dioxide, as well as oxides of iron, zirconium, silicon, manganese, aluminum and cerium and mixtures thereof. Silicates (talc), barium sulfate or zinc stearate may be used as salts. The oxides and salts are used in the form of pigments for skin care and skin protection emulsions and decorative cosmetics. In this case, the particles should have an average diameter of less than 100 nm, preferably 5 to 50 nm and in particular 15 to 30 nm. They can have a spherical shape, but particles may also be used that have an ellipsoid shape or another form deviating from the spherical shape. The pigments can also be present in surface-treated, i.e. hydrophilized or hydrophobized form. Typical examples are coated titanium dioxides, such as e.g. titanium dioxide T 805 (Degussa) or Eusolex® T2000, Eusolex® T, Eusolex® T-ECO, Eusolex® T-Aqua, Eusolex® T-45D (all Merck), Uvinul TiO2 (BASF). In this context, suitable hydro-phobic coating agents are primarily silicones, specifically trialkoxyoctylsilanes or simethicones. In sun protection agents, so-called micro- or nanopigments are preferably used. Preferably, a micronized zinc oxide such as e.g. Z-COTE® or Z-COTE HP1® is used.

In order to prevent the decomposition of certain detergent ingredients catalyzed by heavy metals, substances that complex heavy metals may be used in the macroemulsion according to the present invention. Suitable heavy metal complexing agents are for example the alkali salts of ethylene diamine tetra-acetic acid (EDTA) or nitrilotriacetic acid (NTA) and alkali metal salts of anionic polyelectrolytes such as polymaleates and polysulfonates. A preferred class of complexing agents are the phosphonates, which are included in preferred textile treatment agents in amounts of 0.01 to 2.5% by weight, preferably 0.02 to 2% by weight and in particular 0.03 to 1.5% by weight. Examples of these preferred compounds include in particular organophosphonates such as e.g. 1-hydroxyethane-1,1-diphosphonic acid (HEDP), aminotri(methylene phosphonic acid) (ATMP), diethylene triamine penta(methylene phosphonic acid) (DTPMP or DETPMP) and 2-phosphonobutane-1,2,4-tricarboxylic acid (PBS-AM), which are usually used in the form of their ammonium or alkali metal salts.

The hydrophobic active substances specified above can be used either singularly or in a mixture of two or three or four or even more of the above substances.

In the macroemulsion of the present invention, the amount of the hydrophobic active substance used is 0.1 to 75% by weight, preferably 1 to 50% by weight, more preferred 2 to 20% by weight, particularly preferred 3 to 10% by weight, and most preferred 4 to 7% by weight, relative to the total weight of the oil-in-water macroemulsion composition.

According to a particular and preferred variant, the hydrophobic active substance comprises or consists of a perfume substance or aroma substance or at least one perfume oil or aroma, if the macroemulsion of the present invention is to be used for imparting, i.e. delivering or transferring, a fragrance or aroma, i.e. it is dispersed in the oily or dispersed phase of the macroemulsion of the present invention.

A perfume substance or aroma substance is understood here to mean a compound which is used for the primary purpose of conferring or modifying an odour or flavour. In other words, in order to be considered a perfuming or flavouring substance, such an ingredient must be recognised by a person skilled in the art as being able to at least impart or modify the odour or flavour of a composition in a positive or pleasant way. For the purposes of the present invention, the terms “perfume oil” or “aroma” include a combination of perfuming or flavouring ingredients for modifying or imparting an odour or flavour.

The following specified odoriferous or aroma substances can be used, either as individual substances or in mixtures with at least one other odoriferous or aroma substance, in a large number of fragrance or aroma mixtures, selected from an extensive range of natural and synthetic substances.

Perfume substances or aroma substances which are advantageously suitable for combining are listed for example in S. Arctander, Perfume and Flavor Materials, volumes I and II, Montclair, N.J. 1969, private publication, and/or in H. Surburg, J. Panten, Common Fragrance and Flavor Materials, 6th edition, Wiley-VCH, Weinheim 2016. The following list comprises examples of known odoriferous substances or aroma substances:

Fragrances and perfume oils may be natural odourant mixtures, such as those obtainable from plant sources, examples being extracts of natural raw materials such as essential oils, concretes, absolutes, resins, resinoids, balsams, tinctures such as for example: ambergris tincture; amyris oil; angelica seed oil; angelica root oil; anise oil; valerian oil; basil oil; tree moss absolute; bay oil; artemisia oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; eucalyptus citriodora oil; eucalyptus oil; fennel oil; pine-needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; Litsea cubeba oil; bay leaf oil; mace oil; marjoram oil; mandarin oil; massoia bark oil; mimosa absolute; ambrette oil; musk tincture; muscatel sage oil; nutmeg oil; myrrh absolute; myrrh oil; myrtle oil; clove leaf oil; clove bud oil; neroli oil; olibanum absolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea tree oil; terpentine oil; thyme oil; Tolu balsam; tonka absolute; tuberose absolute; vanilla extract; violet leaf absolute; verbena oil; vetiver oil; juniper berry oil; cognac oil; wormwood oil; wintergreen oil; ylang ylang oil; hyssop oil; civet absolute; cinnamon leaf oil; cinnamon bark oil, and fractions thereof or constituents isolated therefrom;
individual odoriferous substances from the group comprising hydrocarbons, such as for example 3-carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
aliphatic aldehydes and the acetals thereof such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; 1-(1-methoxypropoxy)-(E/Z)-3-hexene;
cycloaliphatic aldehydes such as for example 2,4-dimethyl-3-cyclohexene carbaldehyde; 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde;
aromatic and araliphatic aldehydes such as for example: benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
esters of aliphatic carboxylic acids such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl-isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; methyl-3,7-dimethyl-2,6-octadienoate; 4-methyl-2-pentyl-crotonate;
esters of cyclic alcohols such as for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;
esters of araliphatic alcohols and aliphatic carboxylic acids such as for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenyl-ethyl butyrate; cinnamyl acetate; 2-phenoxyethyl isobutyrate; 4-methoxybenzyl acetate;
esters of cycloaliphatic alcohols such as for example 1-cyclohexylethyl crotonate; esters of cycloaliphatic carboxylic acids such as for example allyl-3-cyclohexyl propionate; allylcyclohexyl oxyacetate; cis- and trans-methyl dihydrojasmonate; cis- and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolane 2-acetate;
aromatic and araliphatic carboxylic acids and the esters thereof such as for example: benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethylbenzoate; ethyl-3-phenyl glycidate; ethyl-3-methyl-3-phenyl glycidate;
aliphatic alcohols such as for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixtures of 3,4,5,6,6-pentamethyl-3,4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic ketones and the oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds such as for example 3-methylthio-hexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
aliphatic nitriles such as for example 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;
acyclic terpene alcohols such as for example: citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
acyclic terpene aldehydes and ketones such as for example geranial; neral; citronellal; 7-hydroxy-3,7-dimethyloctanal; 7-methoxy-3,7-dimethyloctanal; 2,6,10-trimethyl-9-undecenal; geranyl acetone; and the dimethyl and diethyl acetals of geranial, neral, 7-hydroxy-3,7-dimethyloctanal;
cyclic terpene alcohols such as for example: menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;
cyclic terpene aldehydes and ketones such as for example menthone; isomenthone; 8-mercaptomenthan-3-one; carvone; camphor; fenchone; alpha-ionone; beta-ionone; alpha-n-methyl ionone; beta-n-methyl ionone; alpha-isomethyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedryl ketone);
cyclic alcohols such as for example: 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols such as for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers such as for example: cineole; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodeca-hydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones such as for example 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic ketones such as for example 1-(3,3-dimethyl-cyclohexyl)-4-penten-1-one; 2,2-dimethyl-1-(2,4-dimethyl-3-cyclohexene-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl-(2,4-dimethyl-3-cyclohexen-1-yl) ketone;
aromatic hydrocarbons such as for example styrol and diphenyl methane;
araliphatic alcohols such as for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
araliphatic ethers such as for example: 2-phenyl ethyl methyl ether; 2-phenyl ethyl isoamyl ether; 2-phenyl ethyl 1-ethoxyethyl ether; phenylacetaldehyde dimethylacetal; phenylacetaldehyde diethylacetal; hydratropaldehyde dimethylacetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
aromatic and araliphatic ketones such as for example: acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone; nitrogenous aromatic compounds such as for example: 2,4,6-trinitro-1,3-dimethyl-5-tert-butylbenzene; 3,5-dinitro-2,6-dimethyl-4-tert-butyl aceto-phenone; cinnamonitrile; 3-methyl-5-phenyl-2-pentenoic acid nitrile; 3-methyl-5-phenylpentanoic acid nitrile; methyl anthranilate; methyl-N-methyl anthranilate; Schiff bases of methyl anthranilate with 7-hydroxy-3,7-dimethyloctanal, 2-methyl-3-(4-tert-butylphenyl)propanal or 2,4-dimethyl-3-cyclohexene carbaldehyde 6-isopropyl quinoline; 6-isobutyl quinoline; 6-sec-butyl quinoline; 2-(3-phenylpropyl)pyridine; indole; skatole; 2-methoxy-3-isopropylpyrazine; 2-isobutyl-3-methoxypyrazine;
phenols, phenyl ethers and phenyl esters such as for example: estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl acetate;
heterocyclic compounds such as for example: 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones such as for example: 1,4-octanolide; 3-methyl-1,4-octanolide; 1,4-nonanolide; 1,4-decanolide; 8-decen-1,4-olide; 1,4-undecanolide; 1,4-dodecan-olide; 1,5-decanolide; 1,5-dodecanolide; 4-methyl-1,4-decanolide; 1,15-penta-decanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin;
and mixtures of the above substances.

The perfume oil transfer of a laundry perfume heavily depends on the properties of the perfume substance or the aroma substance or the perfume oil or the aroma. Perfume substances or aroma substances with low water solubility and/or high log P value appear to adhere better to cloth. Hence, in a preferred variant of the present invention, the macroemulsion comprises as active hydrophobic substance perfume substances or aroma substances which have a low water solubility and/or a high log P value.

The partition coefficient (P) is the ratio of concentrations of a compound in a mixture of two immiscible solvents at equilibrium. This ratio is therefore a comparison of the solubility of the solute in these two liquids. In the chemical and pharmaceutical sciences, both phases usually are solvents. Most commonly, one of the solvents is water, while the second is hydrophobic such as 1-octanol. Hence the partition coefficient measures how hydrophilic or hydrophobic a chemical substance is.

The log P value is a constant defined in the following manner:

Log P=log 10(Partition Coefficient)

Partition Coefficient,P=[organic]/[aqueous]

wherein [ ] indicates the concentration of solute in the organic and/or aqueous partition.

A negative value for log P means the compound has a higher affinity for the aqueous phase (it is more hydrophilic); when log P=0 the compound is equally partitioned between the lipid and aqueous phases; a positive value for log P denotes a higher concentration in the lipid phase (i.e., the compound is more lipophilic). Log P=1 means there is a 10:1 partitioning in organic:aqueous phases.

Although log P is a constant, its value is dependent on the choice of the organic partitioning solvent.

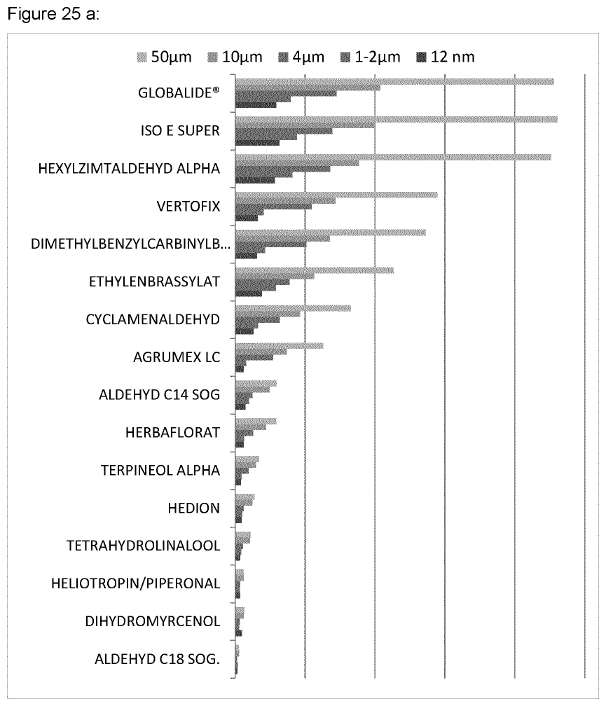

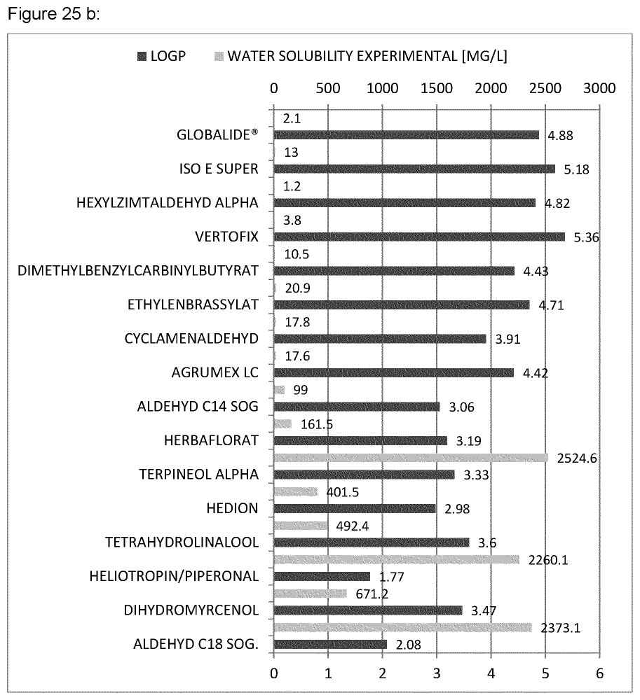

In particular preferred are perfume substances or aroma substances with a water solubility 100 mg/I at room temperature and a log P value of 3.0, based on a water/n-octanol (1:1) system, which seem to be transferred best to the laundry in a washing process and, thus, are recommendable for application in laundry scent or laundry scent booster (see FIGS. 25a and 25b). If the water solubility is >100 mg/I, the perfume or aroma substance(s) is/are washed away with the wash water in the washing/laundry process. On the other hand, a log P value of >3.0 is favourably for the solubility of the perfume or aroma substances in the oily phase and the transfer of the perfume or aroma substances in the washing/laundry process to the clothes.

The log P values of perfume or aroma substances have been reported in many databases, for example the Pomona 92 database, available from Daylight Chemical Information Systems, Inc., Daylight CIS, Irvine, Calif.

In the following table the log P values and the water solubility of some exemplary perfume substances or aroma substances used according to the present invention are specified:

| | | Water solubility |
|---|---|---|
| Compound | log P | [mg/l] |
| DIHYDROMYRCENOL | 3.47 | 671.2 |
| HELIOTROPIN/PIPERONAL | 1.77 | 2260.1 |
| ALDEHYD C18 SOG. | 2.08 | 2373.1 |
| HEDION | 2.98 | 401.5 |
| TETRAHYDROLINALOOL | 3.6 | 492.4 |
| ALDEHYD C14 SOG | 3.06 | 99 |
| HERBAFLORAT | 3.19 | 161.5 |
| TERPINEOL ALPHA | 3.33 | 2524.6 |
| ETHYLENBRASSYLAT | 4.71 | 20.9 |
| CYCLAMENALDEHYD | 3.91 | 17.8 |
| ISO E SUPER | 5.18 | 13 |
| HEXYLZIMTALDEHYD ALPHA | 4.82 | 1.2 |
| GLOBALIDE ® | 4.88 | 2.1 |
| DIMETHYLBENZYLCARBINYLBUTYRAT | 4.43 | 10.5 |
| VERTOFIX | 5.36 | 3.8 |
| AGRUMEX LC | 4.42 | 17.6 |

As it is obvious from FIGS. 25a and 25b, the quantitative transfer and deposition for example of GLOBALIDE®, which has a water solubility of 2.1 mg/I, to/on the clothes in the washing/laundry process is excellent. For perfume or aroma substances, having a water solubility between 100 to 600 mg/I, like aldehyde C18 or higher, the transfer and deposition to/on the clothes, however, is poor.

In perfume or aroma substance macroemulsions according to the present invention, the water solubility of the perfume or aroma substance is preferably 50-100 mg/I, more preferred 20 to 50 mg/I, and most preferred 0 to 20 mg/I (all values at room temperature) and/or the log P value, based on a water/n-octanol (1:1) system, is preferably 3.0 to 3.9, more preferred 3.9 to 4.5, and more preferred>4.5, in order to increase the quantitative transfer and deposition of fragrance or aroma to/on the clothes in a washing/laundry.

If a perfume oil or aroma is used as hydrophobic active substance, than a predominant amount or almost all of the perfume components or aroma components constituting the perfume oil or the aroma should have a high water solubility and/or a low log P value as defined above, in order to improve the quantitative transfer of the fragrance or aroma to the laundry in a washing process.

In perfume or aroma substance macroemulsions of the present invention, the amount of the odoriferous or aroma substance used is 0.1 to 75% by weight, preferably 1 to 50% by weight, more preferred 2 to 20% by weight, particularly preferred 3 to 10% by weight, and most preferred 4 to 7% by weight, relative to the total weight of the oil-in-water macroemulsion composition. Thus, in a preferred variant of the present invention, the odoriferous or aroma substance(s) constitute(s) the oily phase.

The hydrophobic active substance, in particular the perfume or aroma substance, of the macroemulsion according to the present invention may be used in liquid form, either undiluted or diluted with a non-polar solvent immiscible with water.

Specific non-limiting examples of such solvents include for example animal or vegetable oils or fats or their hydrolysates, paraffin oils, silicones, isopropyl myristate, DPG (dipropylene glycol), DPM (dipropylene glycol methyl ether), ethanol, isopropanol, glycol, glycerol derivatives, triethylcitrate, triacetin, benzyl benzoate, MMB (3-Methoxy-3-methyl-1-butanol), Isopar L® (C11-13 Isoparaffin), neononyl acetate, dioctyl adipate, propylene carbonate, ethyl acetoacetate.

Alternatively, the hydrophobic active substance, in particular the perfume or aroma substance, may be—at least partly—encapsulated. The encapsulation can be in the form of microcapsules which have been widely described in the prior art.

The term “capsules” is understood to refer to spherical aggregates comprising at least one solid or liquid core that is enclosed by at least one continuous shell. Here, the hydrophobic active substance can be in the form of macrocapsules with diameters of approximately 0.1 to approximately 5 mm or microcapsules with diameters of approximately 0.0001 to approximately 0.1 mm. The capsules can also have two or more shells of differing composition.

The microcapsules have a core and a microcapsule wall which encapsulates the core. The core comprises at least one of the hydrophobic active substances described above. The nature of the polymeric shell of the microcapsules of the invention can vary. Non-limiting examples of materials from which the wall can be formed include a polymer such as a urea-formaldehyde polymer, a melamine-formaldehyde polymer, a phenolic-formaldehyde polymer, a urea-glutaraldehyde polymer, a melamine-glutaraldehyde polymer, a phenolic-glutaraldehyde polymer, polyurea, polyurethane, polyacrylate, polyamide, polyester, an epoxy cross-linked polymer, a polyfunctional carbodiimide cross-linked polymer, silica, a silica-derived material, polysiloxanes, polyimide, polyvinyl alcohol, polyanhydride, polyolefin, polysulphone, polysaccharide, protein, polylactide (PLA), polyglycolide (PGA), polyorthoester, polyphosphazene, silicone, lipid, modified cellulose, gum, polystyrene and combinations of these materials. Other suitable polymeric materials are ethylene maleic anhydride copolymer, styrene maleic anhydride copolymer, ethylene vinyl acetate copolymer, and lactide glycolide copolymer. Biopolymers derived from alginate, chitosan, collagen, dextran, gelatine and starch can also be used as the encapsulating materials. Preferred encapsulating wall polymers include those formed from isocyanates, acrylates, acrylamides, acrylate-co-acrylamides, hydrogel monomers, sol-gel precursors, gelatine, melamine-formaldehyde or urea-formaldehyde condensates or cross-linked melamine formaldehyde or melamine glyoxal, as well as similar types of aminoplasts.

In particular, the encapsulating wall is polyurea-based and made from for example isocyanate-based monomers and amine-containing crosslinkers such as guanidine carbonate and/or guanazole. Preferred polyurea microcapsules comprise a polyurea wall which is the reaction product of the polymerisation between at least one polyisocyanate comprising at least two isocyanate functional groups and at least one reactant selected from the group consisting of an amine,

Alternatively, the encapsulation shell is polyamine-based and made from for example melamine and formaldehyde or a phenol resin. Preferred melamine-formaldehyde capsule shells consist of different layers of melamine and different linkers from the group of ketones, phenols or aldehydes.

According to another variant, the shell is polyurethane-based and made from for example polyisocyanate and polyol, polyamide, polyester, etc. or may comprise any suitable resin including in particular melamine, glyoxal, polyurea, polyurethane, polyamide, polyester, etc. Suitable resins include the reaction product of an aldehyde and an amine, suitable aldehydes including formaldehyde and glyoxal. Suitable amines include melamine, urea, benzoguanamine, glycoluril and mixtures thereof. Suitable melamines include methylol melamine, methylated methylol melamine, imino melamine and mixtures thereof. Suitable ureas include dimethylol urea, methylated dimethylol urea, urea resorcinol and mixtures thereof.

According to a particular variant, the core-shell microcapsule is a formaldehyde-free capsule.

In a preferred variant according to the first aspect of the present invention, the macroemulsion comprises one part of the total amount of the hydrophobic active substance which is freely dispersed in the aqueous phase, and another part of the total amount of the hydrophobic active substance which is dispersed in an encapsulated form in the aqueous phase.

In a most preferred variant, the macroemulsion comprises one part of a perfume or aroma substance which is freely dispersed in the aqueous phase and another part of the perfume or aroma substance which is dispersed in an encapsulated form in the aqueous phase.

Combining a hydrophobic active substance, in particular a perfume or aroma substance, in a free form and in an encapsulated form has the advantageous effect that, when deposited on a subject or object, for example laundry, the active substance in a free form has an immediate effect, whereas the active substance in an encapsulated form has a delayed effect, because the active substance is released only when mechanical stress is exerted on the microcapsule.

Since macroemulsions are not thermodynamically stable, they do not form spontaneously and require energy input, usually in the form of stirring or shaking of some kind, to mechanically mix the otherwise immiscible phases. A surfactant or emulsifier of some sort is also generally required. This helps form emulsions by reducing the interfacial tension between the two phases, usually by acting as a surfactant or adsorbing onto the interface. A surfactant or emulsifier is a substance which stabilises an emulsion by increasing its kinetic stability and which typically has a polar or hydrophilic (i.e. water-soluble) part and a non-polar (i.e. hydrophobic or lipophilic) part. For this reason, emulsifying agents or surfactants tend to have differing solubility in water and in oil. Emulsifiers which are more soluble in water (and, conversely, less soluble in oil) will generally form oil-in-water emulsions, while emulsifiers which are more soluble in oil will generally form water-in-oil emulsions.

The oil-in-water macroemulsion according to the present invention therefore also comprises at least one surfactant or emulsifying agent.

The choice of said at least one surfactant has a significant impact on the characteristics of the emulsion. In order to retain the stability of the emulsion, the surfactant system has to be carefully adapted.

Surfactants are classified by their hydrophile-lipophile balance (HLB) number, used as a measure of the ratio of these groups and useful in identifying surfactants for oil-and-water emulsification. It is a value between 0 to 20 which defines the affinity of a surfactant for water or oil. HLB numbers are calculated for non-ionic surfactants, and these surfactants have numbers ranging from 0 to 20. HLB numbers over 10 indicate an affinity for water (hydrophilia), and HLB numbers under 10 indicate an affinity for oil (lipophilia).

In order to form and stabilise the oil-in-water macroemulsion according to the present invention, at least one surfactant with an HLB number of between 8 and 18 is used. Preferably, a surfactant with an HLB number between 10 and 14 is used. In a most preferred variant, the macroemulsion of the present invention comprises at least one surfactant with an HLB number of between 11 and 13.

The at least one surfactant or emulsifying agent to be used in implementing the present invention is selected from the group consisting of non-ionic surfactants, anionic surfactants, cationic surfactants and amphoteric surfactants.

Typical examples of non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed eithers or mixed formals, optionally partially oxidized alk(en)yl oligoglycosides or glucuronic acid derivatives, fatty acid-N-alkyl glucamides, protein hydrolysates (in particular wheat-based plant products), polyol fatty acid esters, sucrose esters, sorbitan esters, polysorbates and aminoxides.

Typical anionic surfactants or emulsifiers are aliphatic fatty acids with 12 to 22 carbon atoms, such as e.g. palmitic acid, stearic acid or behenic acid, and dicarboxylic acids with 12 to 22 carbon atoms, such as e.g. azelaic acid or sebacic acid. Typical examples of anionic surfactants are soaps, alkylbenzene sulfonates, alkane sulfonates, olefin sulfonates, alkyl ether sulfonates, glyceryl ether sulfonates, α-methyl ester sulfonates, sulfofatty acids, alkyl sulfates, alkyl ether sulfates, glyceryl ether sulfates, fatty acid ether sulfates, hydroxy mixed ether sulfates, monoglyceride (ether) sulfates, fatty acid amide (ether) sulfates, mono- and dialkylsulfosuccinates, mono- and dialkylsulfosuccinamates, sulfotriglycerides, amide soaps, ether carboxylic acids and salts thereof, fatty acid isethionates, fatty acid sarcosinates, fatty acid taurides, N-acylamino acids, such as e.g. acyl lactylates, acyl tartrates, acyl glutamates and acyl aspartates, alkyl oligoglycoside sulfates, protein fatty acid condensates (in particular wheat-based plant products) and alkyl(ether) phosphates.

Typical examples of cationic surfactants are quaternary ammonium compounds, such as e.g. dimethyl distearyl ammonium chloride, and esterquats, in particular quaternized fatty acid trialkanolamine ester salts.

Moreover, zwitterionic surfactants may be used as surfactants or emulsifiers. Surfactant compounds having in the molecule at least one quaternary ammonium group and at least one carboxylate and one sulfonate group are referred to as zwitterionic surfactants. Particularly suitable zwitterionic surfactants are the so-called betaines, such as the N-alkyl-N,N-dimethyl ammonium glycinates, e.g. coconut alkyldimethyl ammonium glycinate, the N-acylaminopropyl-N,N-dimethyl ammonium glycinates, for example coconut acylaminopropyl dimethyl ammonium glycinate, 2-alkyl-3-carboxylmethyl-3-hydroxyethylimidazolines with 8 to 18 C atoms respectively in the alkyl or acyl group, and coconut acylaminoethyl hydroxyethyl carboxymethyl glycinate. Particularly preferred is the fatty acid amide derivative known by the CTFA name cocamidopropyl betaine. Further suitable emulsifiers are ampholytic surfactants. Ampholytic surfactants are understood to refer to surfactant compounds which, in addition to one C8/18 alkyl or acyl group in the molecule, comprise at least one free amino group and at least one —COOH— or —SO₃H group and are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids with approximately 8 to 18 C atoms respectively in the alkyl group. Particularly preferred ampholytic surfactants are N-coconut alkylaminopropionate, coconut acylaminoethyl aminopropionate and C12/18 acylsarcosine. Typical examples of amphoteric or zwitterionic surfactants are alkyl betaine, alkyl amidobetaines, aminopropionates, aminoglycinates, imidazolinium betaines and sulfobetaines. The above-mentioned surfactants are exclusively known compounds. Typical further examples of particularly suitable surfactants are fatty alcohol polyglycol ether sulfates, monoglyceride sulfates, mono- and/or dialkylsulfosuccinates, fatty acid isethionates, fatty acid sarcosinates, fatty acid taurides, fatty acid glutamates, α-olefin sulfonates, ether carboxylic acids, alkyl oligoglycosides, fatty acid glucamides, alkyl amido betaines, amphoacetals and/or protein fatty acid condensates, the latter preferably based on wheat proteins.

Preferably, the at least one non-ionic surfactant is selected from the group consisting of:

| PN/CAS | | |
|---|---|---|
| non-ionic | Name | Chemical composition |
| 358712 | SymSol PF-3 | water (aqua), pentylene glycol, |
| | | sodium lauryl sulphoacetate, |
| | | sodium oleoyl sarcosinate, |
| | | sodium chloride, |
| | | disodium sulphoacetate, |
| | | sodium oleate, |
| | | sodium sulphate |
| 3332-27-2 | Ammonyx MO | N,N-dimethyldodecylamine-N- |
| | | oxide, lauryldimethylamine-N- |
| | | oxide |
| 68551-12-2 | Berol 175 | laureth-8, Empilan KB-8, |
| | | Walloxen LM 80, Elfapur LM 75 |
| | | S, |
| 68439-46-3 | Berol 266 | C9-11 PARETH-5,5 |
| 26468-86-0 | Berol 840 | 2-ethylhexanol ethoxylat |
| | Empilan KB 10 | ethoxylated C10-C16 alcohols (10 |
| | | EO) |
| 68131-39-5 | Empilan KCL 7 | ethoxylated C12-C15 alcohols, (7 |
| | | EO) |
| | Emulan EL | ethoxylated Ricinus oil (45 EO) |
| | emulsifier | hydrogenated, ethoxylated castor oil |
| | Cremophor CO | (40 EO) |
| | 455 | |
| 61791-12-6 | emulsifier | hydrogenated, ethoxylated castor oil |
| | Cremophor EL | (35 EO) |
| 9005-64-5 | emulsifier | sorbitan ester polyethylene glycol |
| | Tween 20 | ether (20 EO) |
| 69011-36-5 | emulsifier | poly(oxy-1,2-ethanedily), |
| | Trideceth-9 | branched alpha-tridecyl-omega- |
| | | hydroxy |
| 61791-12-6 | FINDET ® AR/ | PEG-40 castor oil |
| | 52 | |
| 68515-73-1 | Glucopon ® 215 | capryl glucoside |
| | UP | |
| | Glucopon ® | alkylpolyglucoside, silicate, |
| | 50 G | sulphate |
| 110615-47-9 | Glucopon ® 600 | C10-C16 alkylpolyglucoside |
| | CSUP | |
| 110615-47-9 | Glucopon ® 650 | coco glucoside |
| | EC | |
| 69011-36-5 | Hoesch T 9 90% | Trideceth-9 |
| 68439-49-6 | Hoesch TG 20 | ceteareth-20 |
| 26183-52-8 | Imbentin AG/100/ | C10 oxoalcohol ethoxylate (8 EO) |
| | 080 | |
| 68439-49-6 | Imbentin AG/ | ceteareth-50 |
| | 168S/500G | |
| 68131-39-5 | Imbentin C/125/ | C12-C15 oxoalcohol ethoxylate |
| | 030 | (3 EO) |
| R-FRA00145 | Lutensol A 8 | C12-C14 oxoalcohol (8 EO) |
| R-FRA00166 | Lutensol XP 140 | isodecanol ethoxylate (14 EO) |
| 69011-36-5 | Marlipal O 13/70 | Trideceth-7 |
| 110615-47-9 | Plantacare 1200 | lauryl glycoside |
| | UP | |
| 141464-42-8 | Plantacare 818 | C18-C16 fatty alcohol glucoside |
| | UP | |
| R-FRA00069 | Plurafac LF 403 | EO/PO oxoalcohol |
| 68140-00-1 | Rewomid C 212 | coco fatty acid monoethanolamide |
| 69011-36-5 | solubilizer | PEG-40 hydrogenated castor oil, |
| | | Trideceth-9, propylene glycol, |
| | | water |
| 2605-79-0 | Tegotens DO | decyldimethylaminoxide |
| R-FRA00058 | Tween 80 V | polyoxyethylene sorbitan fatty |
| | Pharma | acid ester |

Preferably, the at least one anionic surfactants is selected from the group consisting of:

| PN/CAS | | |
|---|---|---|
| Anionic | Name | Chemical composition |
| 14960-06-6 | Deriphat 160 C | sodium lauriminodipropionate |
| 151-21-3 | dodecyl sulphate | dodecyl sulphate, sodium salt |
| | sodium salt | |
| | Empicol EGC 70 | magnesium laureth sulphate |
| 68891-38-3 | Hansanol NS 242 | sodium laureth sulphate |
| | conc | |
| | Hoesch AE 50 | sodium dodecyl benzene sulphonate |
| 85711-69-9 | Hoesch NAS 60 | C13-C17 sec-alkane sulphonate, |
| | | sodium salt |
| 28348-53-0 | Hoesch NCS | sodium isopropyl benzol sulphonate |
| 119415-05-3 | Hostaphat KW 340 | triceteareth-4 phosphate |
| | D | |
| 584-08-7 | Hostapur OSB | olefin sulphonate, sodium salt |
| 68411-30-3 | Marlon A 330 | sodium dodecylbenzene sulphonate |
| 12068-03-0 | Marlon ARL | benzolsulphonic acid, C10-C13 alkyl |
| | | derivates, sodium salt, |
| | | sodium toluenesulphonate |
| 25155-30-0 | Nacconol 90 G | linear sodium alkylbenzene |
| | | sulphonate |
| 110615-47-9 | Plantacare PS 10 | sodium lauryl sulphate, |
| | | lauryl glucoside |
| | Texapon 842 | sodium octylsulphate (C8) |
| 151-21-3 | Texapon K 12 | sodium C12 fatty alcohol sulphate, |
| | Powder | sodium lauryl sulphate |
| 78330-30-0 | Zetesol AO 328 U | sodium pareth sulphate, |
| | | ethoxylated C13-C15 alcohol |
| 68439-50-9 | Zetesol NL-2 | C12-C14 sodium lauryl ether sulphate |

Preferably, the at least one cationic surfactant is selected from the group consisting of:

| PN/CAS | | |
|---|---|---|
| Cationic | Name | Chemical composition |
| 112-02-7 | Dehyquart A-CA | cetrimonium chloride |
| 121-54-0 | Hyamine 1622 | N-benzyl-N,N-dimethyl-N-[4-(1.1.3.3- |
| | | tetramethylbutyl)- |
| | | phenoxyethoxyethyl]ammonium |
| | | chloride, benzetho |
| | Rewoquat SQ 1 | quaternium-80 |
| 91995-81-2 | Rewoquat WE 15 | di-(oleyl carboxyethyl) hydroxyethyl |
| | | methylammonium methosulphate |
| 67-63-0 | Rewoquat WE 18 | dihydrogenated tallowethyl |
| | | hydroxyethylmonium methosulphate |

Preferably, the at least one amphoteric surfactant is selected from the group consisting of:

| PN/CAS | | |
|---|---|---|
| Amphoteric | Name | Chemical composition |
| | Akypo LM-40 | alkyl ether carboxylic acid |
| 68955-55-5 | Ammonyx LO | lauryl myristyl dimethylaminoxide |
| 68140-00-1 | Comperlan 100 | C12-C18 coconut fatty acid |
| | | monoethanolamide |
| 68424-94-2 | Dehyton AB 30 | coco dimethylammonium betaine |
| 61789-40-0 | Dehyton K | cocoyl amide propyldimethyl glycine |
| 26635-93-8 | Genamin O-020 | ethoxylated oleyl amine (2 EO) |
| 61788-90-7 | Genaminox ® CSL | alkyl dimethylaminoxide |
| 61789-40-0 | Hoesch Betain 40 | cocoamidopropyl betaine |

where EO is ethylene oxide and PO is propylene oxide.

In a preferred variant, the at least one surfactant is a non-ionic surfactant or emulsifier. Non-limiting examples of non-ionic surfactants which may be cited include those belonging to the classes of:
addition products of 2 to 30 mol of ethylene oxide and/or 0 to 5 mol of propylene oxide to linear fatty alcohols with 8 to 22 C atoms, fatty acids with 12 to 22 C atoms, alkylphenols with 8 to 15 C atoms in the alkyl group and alkylamines with 8 to 22 carbon atoms in the alkyl radical;
alkyl and/or alkenyl oligoglycosides with 8 to 22 carbon atoms in the alk(en)yl radical and ethoxylated analogs thereof;
addition products of 1 to 15 mol of ethylene oxide to castor oil and/or hardened castor oil;
addition products of 15 to 60 mol of ethylene oxide to castor oil and/or hardened castor oil;
partial esters of glycerol and/or sorbitan with unsaturated, linear or saturated, branched fatty acids with 12 to 22 carbon atoms and/or hydroxycarboxylic acids with 3 to 18 carbon atoms and adducts thereof with 1 to 30 mol of ethylene oxide;
partial esters of polyglycerol (average degree of autocondensation 2 to 8), polyethylene glycol (molecular weight 400 to 5000 g/mol), trimethylolpropane, pentaerythritol, sugar alcohols (e.g. sorbitol), alkyl glycosides (e.g. methyl glycoside, butyl glycoside, lauryl glycoside) and polyglycosides (e.g. cellulose) with saturated and/or unsaturated, linear or branched fatty acids with 12 to 22 carbon atoms and/or hydroxycarboxylic acids with 3 to 18 carbon atoms and adducts thereof with 1 to 30 mol of ethylene oxide;
mixed esters of pentaerythritol, fatty acids, citric acid and fatty alcohol and/or mixed esters of fatty acids with 6 to 22 carbon atoms, methylglucose and polyols, preferably glycerol or polyglycerol, mono, di- and trialkyl phosphate and mono, di- and/or tri-PEG alkyl phosphate and salts thereof;
lanolin alcohols;
polysiloxane-polyalkyl-polyether copolymers or corresponding derivatives; block copolymers, e.g. PEG-30 dipolyhydroxy stearate;
polymer emulsifiers, e.g. of the Pemulen type (TR-1,TR-2) from Goodrich or Cosmedia® SP from Cognis;
polyalkylene glycols and glycerol carbonate.

In the following, particularly suitable emulsifiers are described in further detail:

Alkoxylates. The addition products of ethylene oxide and/or propylene oxide to fatty alcohols, fatty acids, alkylphenols or castor oil constitute known, commercially obtainable products. These are homolog mixtures whose average degree of alkoxylation corresponds to the ratio of the amount of substance of ethylene oxide and/or propylene oxide to the substrates with which the addition reaction is carried out. C12/18 fatty acid mono- and -diesters of addition products of ethylene oxide to glycerol are known as refatting agents for cosmetic preparations.
Alkyl and/or alkenyl oligoglycosides. Alkyl and/or alkenyl oligoglycosides and the production and use thereof are known from the prior art. In particular, they are produced by reacting glucose or oligosaccharides with primary alcohols with 8 to 18 carbon atoms. With respect to the glycoside radical, both monoglycosides in which a cyclic sugar radical is glycosidically bound to the fatty alcohol and oligomeric glycosides with a preferred degree of oligomerization of approximately 8 are suitable. Here, the degree of oligomerization is a statistical mean value on which a homologous distribution that is common for such technical products is based.
Partial glycerides. Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof, which may also secondarily comprise small amounts of triglyceride from the production process. Also suitable are addition products of 1 to 30, preferably 5 to 10 mol of ethylene oxide to the above-mentioned partial glycerides.
Sorbitan esters. Suitable sorbitan esters include sorbitan mono isostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan di hydroxystearate, sorbitan tri hydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Also suitable are addition products of 1 to 30, preferably 5 to 10 mol of ethylene oxide to the above-mentioned sorbitan esters.

Polyglycerol esters. Typical examples of suitable polyglycerol esters are polyglyceryl-di polyhydroxystearate (Dehymuls® PG PH), polyglycerol-3-diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, as well as mixtures thereof. Examples of further suitable polyol esters are mono, di- and tri-esters of trimethylol propane or pentaerythritol, optionally reacted with 1 to 30 mol of ethylene oxide, with lauric acid, coconut fatty acid, tallow fatty acid, palmitic acid, stearic acid, oleic acid, behenic acid and the like.

The above surfactants can be used either individually or in combinations of two, three, four or even more surfactants, preferably in a mixture of two or three surfactants. Due to the different structure and polarity of the hydrophobic active substances, such as for example the perfume substances of a perfume oil, a mixture of two, three, four or even more surfactants is advantageous in order to sufficiently emulsify the hydrophobic active substances and stabilise the macroemulsion. In such a case, the surfactant is a surfactant composition of two, three, four or even more surfactant components. The selection of such a surfactant system depends on the structure and polarity of the hydrophobic active substance to be emulsified and to the remaining components of the oily phase of the macroemulsion.

In a preferred variant of the present invention, a surfactant composition which is a combination of an ionic surfactant, i.e. either an anionic or cationic surfactant, and a non-ionic surfactant, i.e. a combination of surfactants with different HLB values, is used for the preparation and stabilisation of the oil-in-water macroemulsion according to the present invention. For example, a surfactant having an HLB value of 19 can be combined with a surfactant having an HLB value of 2. The ionic surfactant provides the surface of the droplets with a charge. The resulting electrostatic charge prevents the droplets from coalescence. The non-ionic surfactant however prevents diffusion on the interface. The ratio of the ionic surfactant to the non-ionic surfactant, and, thus, the resulting HLB value, is adjusted to the final macroemulsion system.

The macroemulsion of the present invention can best be stabilised by a surfactant composition which exhibits an appropriately adapted HLB number. In another preferred variant, therefore, the macroemulsion according to the present invention comprises a surfactant composition having a final or mean HLB number between 10 and 14, wherein a final or mean HLB number between 11 and 13 stabilises the macroemulsion system best.

A compilation of the most suitable surfactant compositions and their corresponding ratios is shown in FIG. 4.

In addition to the nature and type of surfactant, the concentration of surfactant in the macroemulsion is an important characteristic as compared to conventional microemulsions or solubilised oil solutions.

Typically, an oil-in-water microemulsion according to the prior art requires about 6 to 8% by weight of surfactant, based on the total weight of the emulsion, in order to obtain a dispersed domain diameter ranging from about 1 to 100 nm, usually 10 to 50 nm.

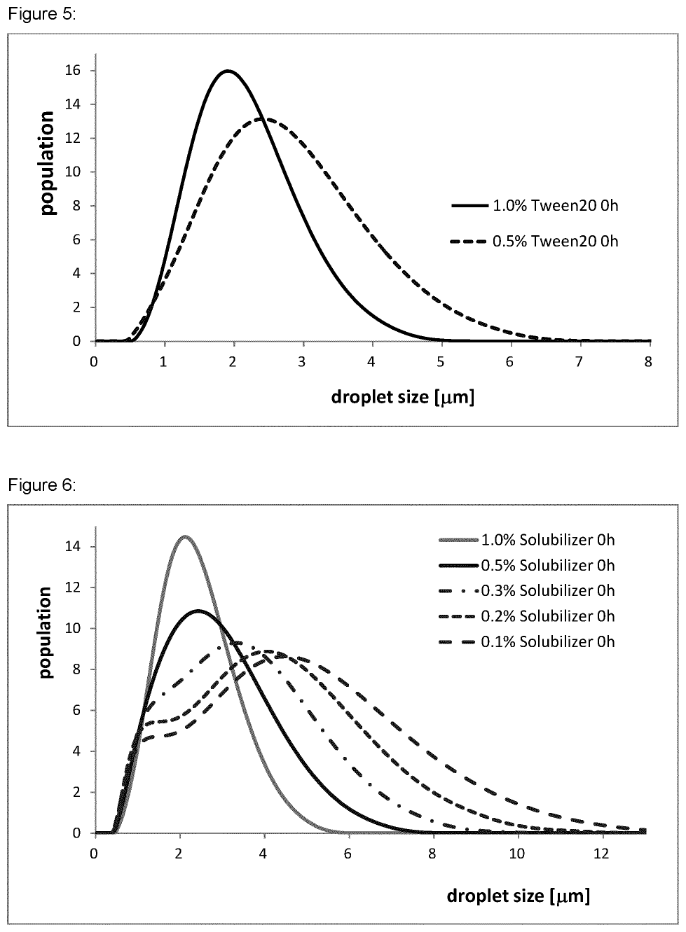

On the other hand, charging, active transfer of the hydrophobic active substance and improved deposition of the hydrophobic active substance require large emulsion droplet sizes. Large droplet sizes in an emulsion can only be obtained using low concentrations of surfactant. A low(er) surfactant concentration significantly increases the droplet size of the macroemulsion. Since active transfer and deposition of the hydrophobic active substance depends on the droplet size, and the droplet size is defined by the surfactant concentration, the amount of surfactant is reduced to just the minimum necessary to formulate a stable macroemulsion (see FIGS. 5 and 6).

The macroemulsion of the present invention therefore comprises from 0.4 to 25% by weight of surfactant or emulsifying agent, preferably from 1 to 20% by weight, more preferably 4 to 15% by weight and most preferably from 8 to 12% by weight, based on the total weight of the oily or dispersed phase or from 0.004 to 18% by weight of surfactant or emulsifying agent, preferably from 0.01 to 10% by weight, more preferably from 0.08 to 3% by weight, particularly preferred from 0.2 to 1.2% by weight, and most preferred from 0.3 to 0.9% by weight, based on the total weight of the macroemulsion. The stability of the macroemulsion can be retained at these surfactant concentrations.

Unlike a conventional microemulsion formulation, which is more stable, the macroemulsion of the present invention is broken up when it is diluted with water, for example in a laundry or cleaning process, and the hydrophobic active substance, in particular the perfume or aroma substance, is deposited on the subject or object, i.e. on fabrics, rather than in the washing or rinsing water.

At the aforementioned surfactant concentrations, a macroemulsion with an average oil droplet size of more than 1 μm, preferably from 2 to 60 μm, can be obtained. A macroemulsion with an average oil droplet size from 4 to 45 μm is particularly preferred, and a macroemulsion with an average oil droplet size from 10 to 30 μm is even more preferred. A macroemulsion with an average oil droplet size of <30 still has a good shelf life stability, while the perfume oil transfer to the clothes increases with the size of the droplets. Therefore, a macroemulsion with an oil droplet size from 10 to 30 μm or even higher is characterised by a good shelf life as well as a favourable transfer of the hydrophobic active substance.

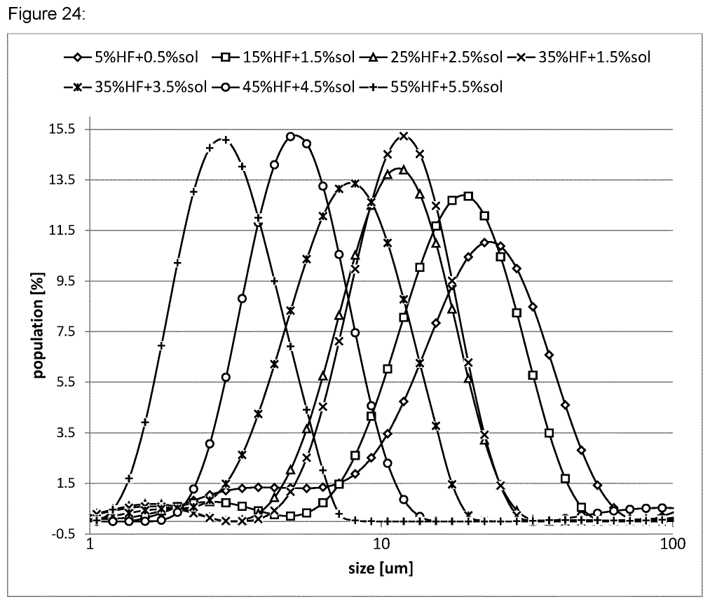

The droplet size decreases with rising the concentration of the hydrophobic active substance, for example perfume substance or aroma substance, even when the surfactant concentration is increased accordingly by the same ratio (see FIG. 24). Hence, the ratio of surfactant or emulsifying agent to hydrophobic active substance has to be adjusted accordingly in order to obtain appropriate droplet size in order to ensure a quantitative transfer and deposition of perfume oil or aroma to/on the laundry in the washing process. The ratio between surfactant or emulsifying agent to the hydrophobic active substance is from 0.4:99.6 to 25:75, preferably from 1:99 to 20 to 80, more preferably from 4:96 to 15:85, and most preferred from 8:92 to 12:88.

The oil-in-water macroemulsion according to the present invention additionally comprises at least one stabiliser. Due to the low surfactant concentration, the stabiliser is an essential component, since it stabilises the macroemulsion and prevents it from degrading into phase separation (creaming, sedimentation), Ostwald ripening, aggregation processes (flocculation, coagulation, coalescence) or phase inversion.

The stabiliser used in accordance with the present invention serves as a thickening agent or thickener and imbues the macroemulsion with a favourable rheological profile. A thickening agent or thickener is a substance which can increase the viscosity of a liquid without substantially changing its other properties. The stability of a macroemulsion depends directly on its viscosity. The higher the viscosity, the greater the stability of the macroemulsion.

By using a stabiliser, i.e. a thickener, the migration velocity of the oil droplets in the macroemulsion is reduced and, thus, all emulsion breaking processes are decelerated. The macroemulsion of the present invention is thus kinetically stabilised. When diluted with water, for example in a washing or cleaning process, the thickener concentration of the macroemulsion is reduced. Thus, the migration velocity of the oil droplets increases, the kinetic stability of the macroemulsion lapses suddenly and the droplets begin to coagulate and are deposited on the subject or object, since the droplets cannot longer be kept apart to distances.

Hence, the thickener on the one hand kinetically stabilises the macroemulsion on emulsion formation and storage and on the other hand destabilises the macroemulsion upon application, for example when the macroemulsion is diluted with water and the concentration of the thickener decreases.

In accordance with the present invention, a large variety of thickeners can be used in combination with different surfactants in order to stabilise the macroemulsion according to the first aspect of the present invention. Of the above stabilisers, thixotropic thickeners are best at stabilising the macroemulsion while allowing themselves to be rinsed at a certain shear force. In rheology, shear thinning is the non-Newtonian behaviour of fluids whose viscosity decreases under shear strain. In a preferred variant, the macroemulsions therefore comprise at least one thixotropic thickener as the stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein. However, modified natural substances such as modified starches and modified celluloses, here one can mention for example carboxymethylcellulose and other cellulose ethers, hydroxyethyl- and -propyl cellulose and locust bean gum ethers, can also be used as thickeners.

Examples of polyacrylic and polymethacrylic thickeners include the high molecular weight homopolymers of acrylic acid crosslinked with a polyalkenyl polyether, in particular an allyl ether of saccharose, pentaerythritol or propylene (INCI name according to the “International Dictionary of Cosmetic Ingredients” of “The Cosmetic, Toiletry and Fragrance Association (CTFA)”: Carbomer), which are also referred to as carboxyvinyl polymers. Such polyacrylic acids include the products available from 3V Sigma under the name Polygel®, e.g. Polygel DA, and from B. F. Goodrich under the name Carbopol®, e.g. Carbopol 940 (molecular weight approx. 4,000,000 g/mol), Carbopol 941 (molecular weight approx. 1,250,000) or Carbopol 934 (molecular weight approx. 3,000,000 g/mol). Also included herein are the following acrylic acid copolymers: (i) copolymers of two or more monomers from the group of acrylic acid, methacrylic acid and their simple esters, preferably formed with C1-4 alkanols (INCI: Acrylates Copolymer), including for example the copolymers of methacrylic acid, butyl acrylate and methyl methacrylate (CAS name according to Chemical Abstracts Service: 25035-69-2) or of butyl acrylate and methyl methacrylate (CAS 25852-37-3) and which for example are available from Rohm and Haas under the brand names Aculyn® and Acusol® and from Degussa (Goldschmidt) under the name Tego® Polymer, e.g. the anionic non-associative polymers Aculyn 22, Aculyn 28, Aculyn 33 (crosslinked), Acusol 810, Acusol 820, Acusol 823 and Acusol 830 (CAS 25852-37-3); (ii) crosslinked high molecular weight acrylic acid co-polymers, including for example the copolymers, crosslinked with an allyl ether of saccharose or pentaerythritol, of C10-30 alkyl acrylates with one or more monomers from the group of acrylic acid, methacrylic acid and their simple esters, preferably formed with C1-4 alkanols (INCI: Acrylates/C10-30 Alkyl Acrylate Crosspolymer), which are obtainable for example from B. F. Goodrich under the name Carbopol®, e.g. the hydrophobized Carbopol ETD 2623, Carbopol 1382 (INCI: Acrylates/C10-30 Alkyl Acrylate Crosspolymer) and Carbopol Aqua 30 (formerly Carbopol EX 473).

A further polymeric thicker that is preferably to be used is xanthan gum, a microbial anionic heteropolysaccharide that is produced by Xanthomonas campestris and several other species under aerobic conditions and has a molecular weight of 2 to 15 million g/mol. Xanthan is composed of a chain of β-1,4-bound glucose (cellulose) with side chains. The structure of the subgroups is composed of glucose, mannose, glucuronic acid, acetate and pyruvate, wherein the number of pyruvate groups determines the viscosity of the xanthan gum. In particular, a fatty alcohol is also suitable as a thickener. Fatty alcohols can be branched or unbranched and can be of native or petrochemical origin. Preferred fatty alcohols have a C chain length of 10 to 20, and preferably 12 to 18 C atoms. Preferably, mixtures of different C chain lengths such as tallow fatty alcohol or coconut fatty alcohol are used. Examples are Lorol® special (C12-14 ROH) or Lorol® technical (C12-18 ROH) (both from Cognis). Preferred liquid detergents and cleaning agents comprise 0.01 to 3% by weight and preferably 0.1 to 1 wt % of thickener based on the total amount of agent. In this case, the amount of thickener used depends on the type of thickener and the desired degree of thickening.

The stabiliser in the oil-in-water macroemulsion also allows the successful stable suspension of microcapsules comprising a hydrophobic or lipophilic active substance, as is the case in the laundry or cleaning sector. Due to the higher Newtonian viscosity the microcapsules remain dispersed in the macroemulsion formulation and would not sediment as compared to microemulsions according to the prior art.

In applications such as laundry perfumes or fabric softeners, however, the viscosity is restricted by the suction of the conditioner tray in the washing machine. If the viscosity of the macroemulsion is too high, or the solubility of the macroemulsion in water is too low, residual amounts will remain in the conditioner tray or on the surface to be treated, hence the concentration of stabiliser has to be carefully adapted.

The oil-in-water macroemulsion advantageously comprises up to 1% by weight of stabiliser, i.e. thickener, preferably in the range of 0.1 to 0.9% by weight and more preferably in the range of 0.4 to 0.6% by weight, based on the total amount of the macroemulsion.

The concentration of the stabiliser is adapted in such a way that the viscosity of the macroemulsion as a whole is no higher than 1000 m·Pas at a temperature in the range of 20 to 25° C. Preferably, the oil-in-water macroemulsion of the present invention has a viscosity in the range of 700 to 1000 m·Pas, more preferably a viscosity in the range of 800 to 900 mPa·s, at a temperature in the range of 20 to 25° C.

The viscosity can be measured by the usual standard methods using a Brookfield viscosimeter RV/RS/MARS available from Thermo Electron (Karlsruhe) GmbH.

In addition to the components described above, in particular the hydrophobic active substances, the oil-in-water macroemulsion according to the present invention can also optionally comprise additives and/or adjuvants. Preferably, the additives and/or adjuvants are selected from the group consisting of colorants, preservatives, deposition aids, etc., without being limited to such components.

The deposition aid is used to aid in the deposition of microcapsules on surfaces such as fabric, hair or skin. Examples of deposition aids include anionically, cationically, non-ionically or amphoterically water-soluble polymers.

The macroemulsion can comprise up to 10% by weight, preferably less than 5% by weight, even more preferably up to 1% by weight and most preferably up to 0.1% by weight of the additives and/or adjuvants, based on the total weight of the macroemulsion composition.

The oil-in-water macroemulsion composition according to the present invention advantageously comprises or consists of the following components:

25 to 99.9% by weight, preferably 50 to 99% by weight, more preferably 80 to 98% by weight, particular preferred 90 to 97% by weight, most preferably 93 to 96% by weight, of the aqueous phase;
0.1 to 75% by weight, preferably 1 to 50% by weight, more preferably 2 to 20% by weight, particular preferred 3 to 10% by weight, most preferably 4 to 7% by weight, of the oily phase comprising at least one hydrophobic active substance, in particular a perfume or aroma substance;
0.004 to 18% by weight, preferably 0.01 to 10% by weight, more preferably 0.8 to 3% by weight, particularly preferred 0.2 to 1.2% by weight, most preferably 0.3 to 0.9% by weight of surfactant;
up to 1% by weight, preferably 0.1 to 0.9% by weight of stabiliser; and optionally up to 1% by weight, preferably 0.1 to 0.9% by weight of additives and/or adjuvants;
based on the total weight of the macroemulsion.

The oil-in-water macroemulsion according to the present invention is characterised by average droplet sizes of more than 1 μm, preferably from 2 to 60 μm, more preferably from 4 to 45 μm and most preferred from 10 to 30 μm, and by a reduction in the concentration of surfactant.

A significant enhancement of hydrophobic active substance transfer, in particular a significant enhancement of perfume or aroma substance transfer, as compared to microemulsions according to the prior art was confirmed.

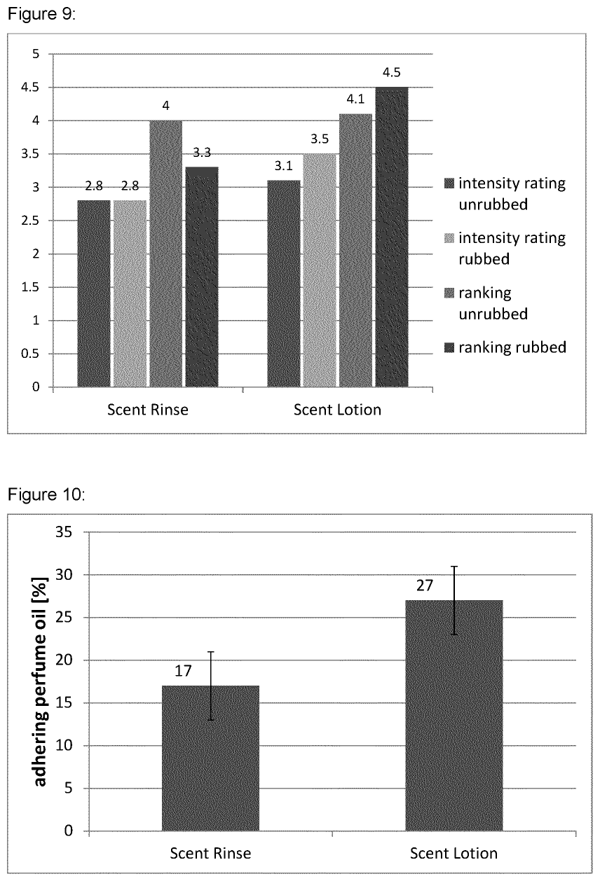

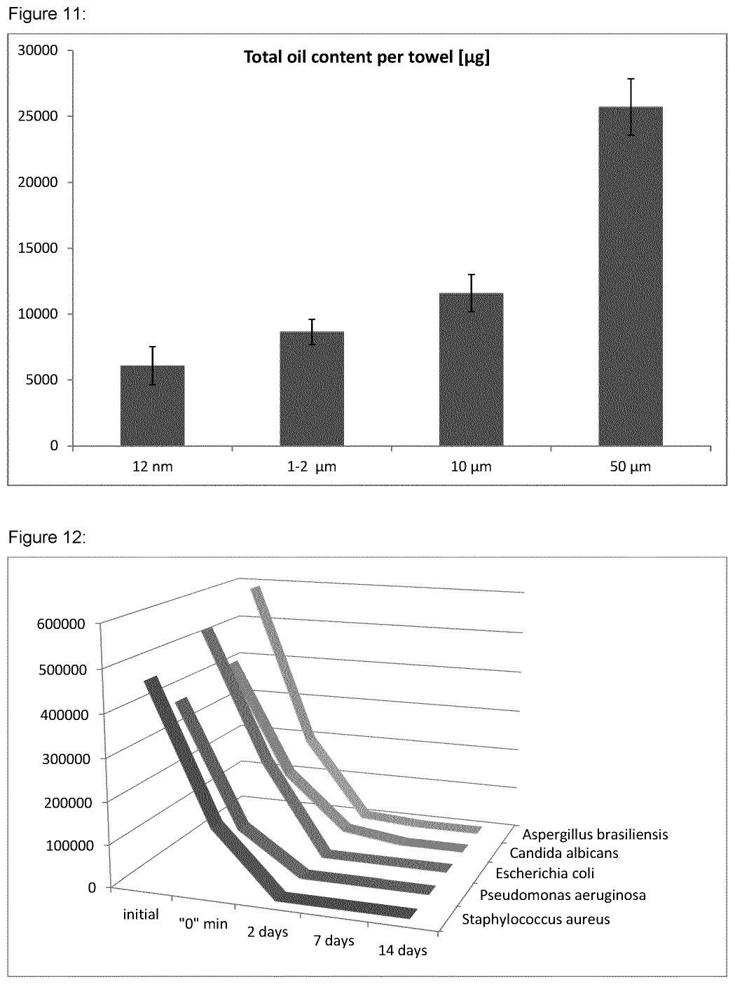

As shown by the sensory test for a scent booster composition, the oil-in-water macroemulsion (scent lotion) according to the present invention was perceived as more intense than the emulsion (scent rinse) according to the prior art (see FIGS. 8 and 9). These results correlate with an analytical quantification which confirms an improved quantitative transfer and deposition of perfume oil to/on clothes for an oil-in-water macroemulsion (scent lotion) as compared to an emulsion (scent rinse) according to the prior art (see FIGS. 10 and 11). Remarkably, the absolute amount of adhering perfume substances correlates with the droplet size of the emulsion.

In a second aspect, the present invention pertains to a method for preparing an oil-in-water macroemulsion. The method comprises the steps of:
(1-i) providing an aqueous phase by mixing and dissolving at least one stabiliser and optionally at least one other additive and/or adjuvant in an aqueous solution;
(1-ii) providing an oily phase by mixing and dissolving at least one hydrophobic active substance and/or optionally at least one hydrophobic active substance in a microcapsule form, and at least one surfactant in an oily solution; and
(1-iii) dispersing the oily phase in the aqueous phase by stirring, shaking, pressing or otherwise inducing sheer forces, to obtain an oil-in-water macroemulsion;
   or
(2-i) providing an aqueous phase by mixing and dissolving at least one surfactant, at least one stabiliser and optionally at least one other additive and/or adjuvant in an aqueous solution;
(2-ii) providing an oily phase by mixing and dissolving at least one hydrophobic active substance and/or optionally at least one hydrophobic active substance in a microcapsule form in an oily solution; and
(2-iii) dispersing the oily phase in the aqueous phase by stirring, shaking, pressing or otherwise inducing sheer forces, to obtain a macroemulsion;
   wherein the amount of the surfactant is from 0.4 to 25% by weight, based on the total weight of the oily phase.

With regard to the ingredients for preparing an oil-in-water macroemulsion and their preferred variants, the same applies as has been described above with regard to the oil-in-water macroemulsion.

The oil-in-water macroemulsion can be produced by emulsifying methods using typical techniques which are well known in the prior art in the field of emulsions.

Depending on the type of surfactant used for preparing the oil-in-water macroemulsion, in the method according to the present invention the at least one surfactant can either mixed and dissolved in the aqueous phase or alternatively in the oily phase. Preferably, the at least one surfactant is mixed and dissolved in the oily phase.

Once the aqueous or continuous phase and the oily phase have been provided, the oily phase is dispersed in the aqueous phase by stirring, shaking, pressing or otherwise inducing sheer forces. The droplet size of monodisperse emulsions is usually adapted using a colloid mill, ball mill, homogeniser valve or an Ultra-Turrax®. In order to prepare the oil-in-water macroemulsion according to the present invention, only simple dispersing devices such as an agitator, blade stirrer and/or speed mixer are used. These dispersing devices have the advantage that they can be easily scaled up.

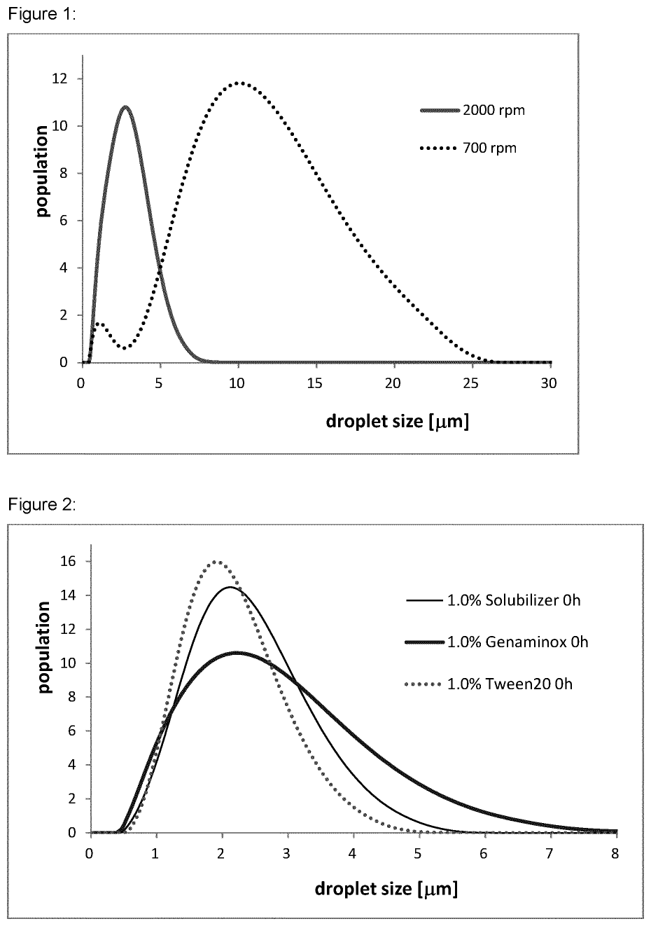

As already described above, the active transfer and deposition of the hydrophobic active substance depends on the droplet size. At low surfactant concentrations, the shear forces of the dispersing method affect the droplet size of the emulsion. In a preferred variant of the method, the emulsion is thus formed with a blade stirrer at a stirring or shaking rate of 500 to 2000 rpm. Most preferably, the emulsion is formed at a stirring or shaking rate of 700 to 1000 rpm. Alternatively, the emulsion is formed with a Ystral stirrer at minimum stirring rate, preferably at a stirring rate of less than 300 rpm. This emulsification treatment adapts the droplet size of the emulsified particles to a diameter of more than 10 μm, as shown in FIG. 1.

Due to the excellent characteristics of the oil-in-water macroemulsion according to the present invention, the present invention also pertains to a method for providing, transferring and depositing a hydrophobic active substance on the surface of a subject or object. In this method, the oil-in-water macroemulsion according to the present invention, which is in particular a perfume or aroma macroemulsion, is provided and brought in contact with the surface of a subject or object, thereby transferring and depositing the hydrophobic active substance.

By means of such a method, a hydrophobic active substance of the macroemulsion can be transferred and deposited on skin, hair, beard, fur or fabrics, clothes, surfaces, natural fibres such as cotton, Akon, kapok, flax, hemp, jute, sunn, kenaf, ramie, sisal, manila hemp, alfa grass, coir, wool, fur, silk, synthetic fibres such as modified cellulose, plant protein fibres, paper fibres, rubber, alginate, casein, polyester, polyamide, polyacryl nitrile, polypropylene, polyethylene, polyvinylchloride, elastane, wood, food, cosmetics, pharmaceuticals and pet foods, in order to provide a sensory effect or other benefits.

In a preferred variant, the method for providing, transferring and depositing a hydrophobic active substance is applied in a washing or rinsing process, in particular a laundry process. The hydrophobic active substance is then preferably a perfume or aroma substance or a perfume oil or aroma or other substance for laundry treatment such as UV-active substances, optical brighteners, drape and form control agents, smoothness agents, static control agents, wrinkle control agents, colour maintenance agents, colour restoring/rejuvenating agents, anti-fading agents, etc.

In another aspect, the present invention also pertains to the use of the oil-in-water macroemulsion according to the present invention in a perfume, flavouring, active skin-product ingredients, active pharmaceutical ingredients, dyes, UV-active substances, optical brighteners, bodying agents, drape and form control agents, smoothness agents, static control agents, wrinkle control agents, sanitising agents, disinfecting agents, germ control agents, mould control agents, mildew control agents, antiviral agents, antimicrobials, drying agents, stain resistance agents, soil release agents, malodour control agents, fabric freshening agents, dye fixatives, colour maintenance agents, colour restoring/rejuvenating agents, anti-fading agents, anti-abrasion agents, wear resistance agents, fabric integrity agents, anti-wear agents, rinsing aids, UV protection agents, sun fade inhibitors, insect repellents, anti-allergenic agents, flame retardants, water-proofing agents, fabric softening agents, shrinkage resistance agents, stretch resistance agents and mixtures thereof.

In yet another aspect, the present invention also pertains to the use of the oil-in-water macroemulsion according to the present invention for preparing consumer products. The oil-in-water macroemulsions of the present invention are suitable for use, without limitation, in the following applications: foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, scent boosters, pharmaceuticals and pet foods.

In a preferred variant, the oil-in-water macroemulsion is used for preparing cosmetics, personal care products, homecare products, in particular liquid detergents, all-purpose cleaners, laundry and cleaning agents, fabric softeners, scent boosters, for conferring, improving or modifying the perfume or aroma properties of said products.

The proportion of oil-in-water emulsion added to the above products varies in accordance with the nature of the product and/or the particular organoleptic or other effect which is to be achieved. Typically, 0.05% to 2% by weight of the oil-in-water emulsion of the invention can be added to the final composition.

In a final aspect, the present invention relates to consumer products which comprise or consist of the oil-in-water macroemulsion of the present invention, such as foods, cosmetics, personal care products, in particular skin cleaning products, shampoos, rinse-off conditioners, deodorants, antiperspirants, body lotions, homecare products, in particular liquid detergents, all-purpose cleaners, laundry and cleaning agents, fabric softeners, laundry scent boosters, pharmaceuticals and pet foods, which also form part of the present invention.

The present invention is described below in more detail by way of examples. The present invention is not limited by these examples. Quantities are given in % by weight unless otherwise stated.

### EXAMPLES

Oil-in-water macroemulsions (hereinafter also referred to as “scent lotions”) in accordance with the present invention, with droplet sizes of 2 to 100 μm, were prepared by reducing the stirring speed (FIG. 1), varying the surfactant (FIGS. 2 to 4) and reducing the surfactant concentration (FIGS. 5 to 7) and following the procedures described below in Example 1 to 3.

The perfume oil delivery of the scent lotion and of a conventional scent rinse to pieces of cloth in a typical washing process was compared by two sensory panels (FIGS. 8 and 9) and by analytical SDE/GCMS (FIGS. 10 and 11). The conventional scent rinse is a conventional laundry perfume with a surfactant concentration of at least the concentration of the dispersed phase, giving a microemulsion (according to the prior art). Both investigation techniques confirmed a significant enhancement of perfume oil transfer in favour of the scent lotion. Remarkably, the absolute amount of adhering perfume oil appears to correlate with the droplet size of the emulsion (FIG. 11).

### Technical Aspects

1. Methods and Conditions

In the emulsion preparation process, an oil-in-water macroemulsion (scent lotion) is essentially formed by dispersing the hydrophobic active substance(s) in an aqueous medium. Droplet sizes in the micrometre range are obtained using comparatively low surfactant concentrations (less than 17% of the perfume oil). Since low surfactant concentrations trigger Ostwald ripening, the emulsion is stabilised by a thickener. The droplet size of the monodisperse emulsion is adapted using a simple dispersing device such as an agitator, blade stirrer and/or speed mixer. This dispersing device can be easily scaled up.

At low surfactant concentrations, the shear forces of the dispersing method affect the droplet size of the emulsion (FIG. 1).

In the characterisation process, the distribution of particle sizes was determined by dynamic light scattering (DLS) using a Mastersizer 2000 (produced by Malvern). Each value represents an average of 10000 snapshots and was measured as a triplicate. The corresponding calculation is based on Mie theory. All measurements were taken under a shading of 10 to 20%. Characteristic values for each measurement (average diameter: 10% of all particles have a diameter of less than d_{(0.1)}; 50% of all particles have a diameter of less than d_{(0.5)}; 90% of all particles have a diameter of less than d_{(0.9)}) are shown in FIG. 1.

FIG. 1 shows the influence of the stirring speed on the droplet size of the oil-in-water emulsion. The surfactant was a solubilizer (1.0%) consisting of 50% PEG-40 hydrogenated castor oil (90%), 45% Trideceth-9 and 5% propylene glycol. The stirring speeds were 2000 rpm (solid curve) and 700 rpm (dotted curve). The droplet sizes were 2.78 μm (d_{(0.1)} 0.98 μm, d_{(0.5)} 2.25 μm, d_{(0.9)} 4.19 μm) at 2000 rpm and 1.10 μm and 10.13 μm (d_{(0.1)} 1.50 μm, d_{(0.5)} 8.84 μm, d_{(0.9)} 15.87 μm) at 700 rpm. No significant changes were observed when the droplet size measurements were repeated under comparable conditions 24 hours later.

2. Preparation Methods

In Example 1, a beaker was supplied with water (90.2 g) in order to prepare 100 g of scent lotion. While stirring with an agitator (5 cm) at 500 rpm, xanthan gum (Keltrol® RD, 700 mg) was carefully added and mixed in for 30 minutes at 1000 rpm. Parmetol® N 20 (100 mg) and a surfactant (Tween 20, 1000 mg) were added to the mixture and stirred until a homogenous solution was obtained. The stirring speed was increased to 2000 rpm, and a perfume oil (HAPPY FEELING, 8.00 g) was dispersed in the solution over a period of 10 minutes.

In Example 2, a solution of xanthan gum (Keltrol® RD, 1000 mg) and Parmetol® N 20 (100 mg) in water (90.80 g) was premixed over 30 minutes at 1000 rpm. The stirring rate was then reduced to 700 rpm, and a solution of a mixture of surfactants (50% PEG-40 hydrogenated castor oil (90%), 45% Trideceth-9 and 5% propylene glycol, water, 100 mg) (hereinafter also referred to as “solubilizer”), in perfume oil (Marrakesh, 8.00 g) was dispersed in the mixture and stirred for another 15 minutes, until a homogenous emulsion was obtained.

In Example 3, a surfactant (Genaminox®, 750 mg) was mixed into a blend of water (92.25 g), xanthan gum (Keltrol® RD, 700 mg) and Parmetol® N 20 (100 mg) at 3500 rpm for 15 seconds in a Speedmixer, until the solution was homogenous. A perfume oil (JOYABLE, 6.00 g) was added and dispersed in the viscous solution for five minutes at 3500 rpm. Solid particles (in this case, 200 mg of a capsule slurry) were added to the completed scent lotion and mixed in for one minute at 3500 rpm, to obtain a homogenous suspension.

3. The Thickener

The thickener is an essential part of the scent lotion, as it stabilises the macroemulsion against Ostwald ripening (see Comparative Example 1). The stability of a scent lotion depends directly on its viscosity. If its viscosity is too high, or its solubility in water is too low, residual amounts will remain in the conditioner tray (see Comparative Example 2).

In Comparative Example 1, a solution of a solubilizer (mixture of surfactants 50% PEG-40 hydrogenated castor oil (90%), 45% Trideceth-9 and 5% propylene glycol) (1 g) in a perfume oil (HAPPY FEELING, 6 g) was dispersed at 2000 rpm using a blade stirrer in an aqueous solution of Esaflor™ HM 22 (0.5 g) and Parmetol® N 20 (0.1 g) in water (91.9 g) for 15 minutes. After a few hours, perfume oil droplets began to float to the surface. After just one day, the phases had completely separated.

In Comparative Example 2, a solution of a solubilizer (mixture of surfactants 50% PEG-40 hydrogenated castor oil (90%), 45% Trideceth-9 and 5% propylene glycol) (1 g) in a perfume oil (HAPPY FEELING, 6 g) was dispersed at 2000 rpm using a blade stirrer in an aqueous solution of Keltrol® RD (1 g in 91.9 g of water) and Parmetol® N 20 (0.1 g) for 15 minutes. Once homogenisation was complete, the viscosity of the scent lotion started to increase. After just one hour, the emulsion had thickened so much that it was considered more of a gel than a lotion. When this gel was provided to the conditioner tray and rinsed, most of it remained as a residual amount in the tray.

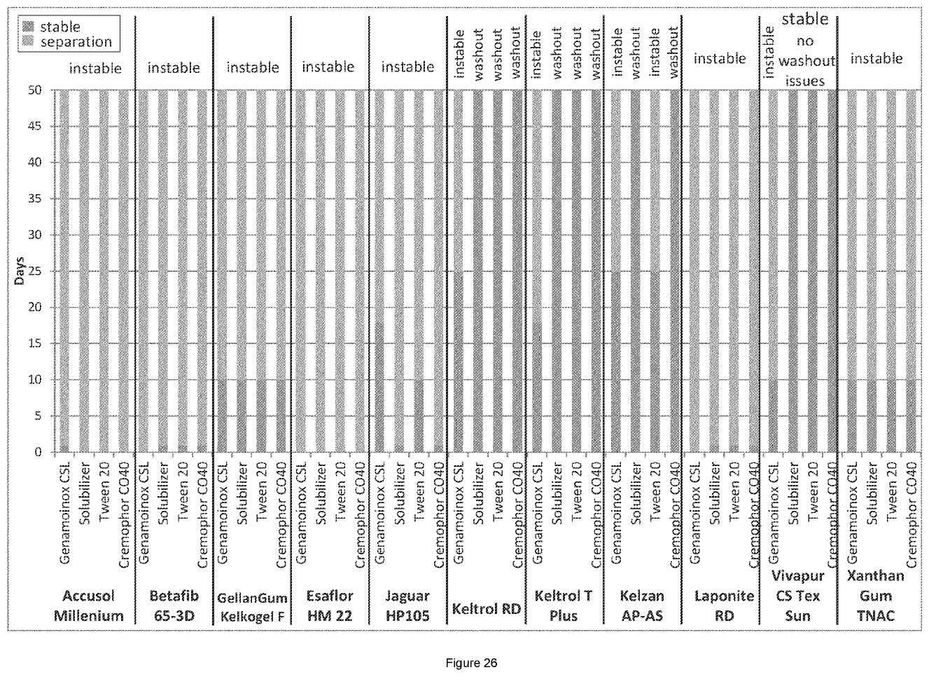

A variety of thickeners were used in combination with different surfactants in order to evaluate their stabilising potential. The results are shown in FIG. 26.

FIG. 26 is a comparison of the stability of scent lotions prepared from different thickeners. For preparing these samples, a solution of the respective surfactant (1 g) in a perfume oil (Marrakesh, 6 g) was dispersed at 2000 rpm using a blade stirrer in an aqueous solution of each thickener at a concentration of 1.0% (except for Esaflor™, which was applied at a lower concentration of only 0.54%) for 30 minutes. All the scent lotions were prepared on the same day, and their stability was evaluated after 10, 18, 25 and 49 days of storage at 50° C.

The thickener and its concentration have to be chosen carefully. Thixotropic thickeners are best at stabilising an emulsion while allowing themselves to be rinsed at a certain shear force. In order to determine the maximum viscosity of a scent lotion for applications in a conditioner tray, rinsing experiments were correlated with the respective viscosity of each sample. In a first experiment, aqueous solutions (18 ml) of a Newtonian thickener (Natrosol™) were rinsed through the conditioner tray of a regular homecare washing machine (a Miele front loader). The concentration of the thickener was increased until the purge process could no longer be completed. Rheological investigation of the thickened solutions showed that the viscosity of the formulation as a whole cannot be higher than 800 to 1000 m·Pas (18 ml of a 0.6% aqueous solution of Natrosol™)

4. The Surfactant

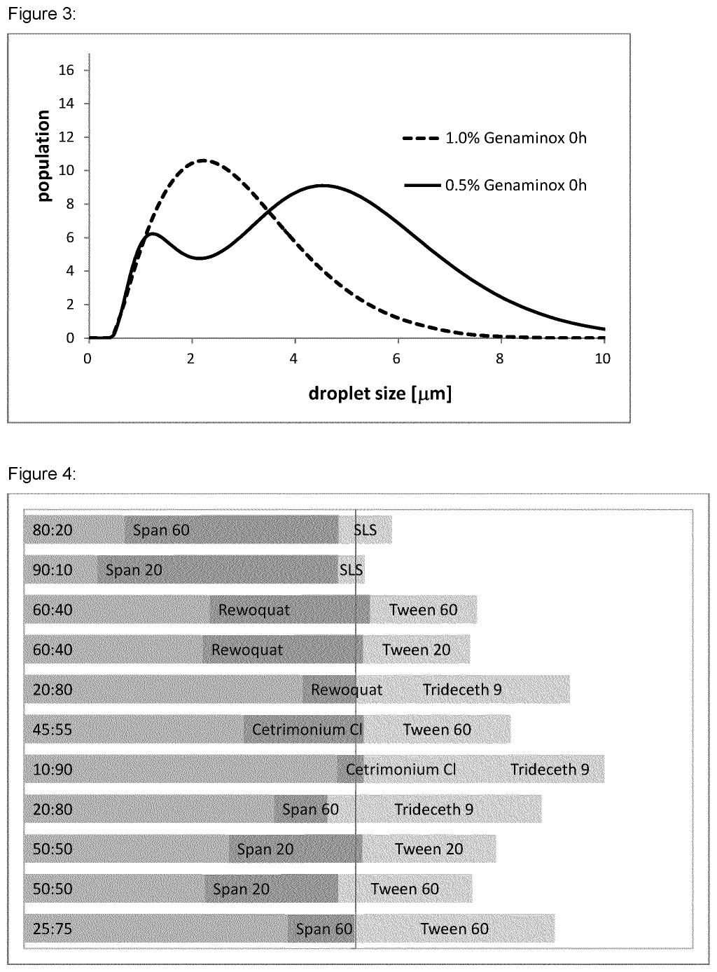

The choice of surfactant has a significant impact on the characteristics of an emulsion (FIGS. 2 and 3). Large droplet sizes can only be obtained using low surfactant concentrations. In order to retain the stability of the emulsion, the surfactant system has to be carefully adapted.

FIG. 2 shows emulsions with different surfactants, wherein all the emulsions were prepared at 2000 rpm using a surfactant concentration of 1.0%. The droplet sizes were as follows: solubilizer (grey curve) 2.78 μm (d_{(0.1)} 0.98 μm, d_{(0.5)} 2.25 μm, d_{(0.9)} 4.19 μm); Genaminox® (black curve) 1.92 μm (d_{(0.1)} 1.10 μm, d_{(0.5)} 1.84 μm, d_{(0.9)} 2.90 μm); Tween 20 (dotted curve) 1.92 μm (d_{(0.1)} 1.12 μm, d_{(0.5)} 1.84 μm, d_{(0.9)} 2.86 μm). No significant changes were observed when the droplet size measurements were repeated under comparable conditions 24 hours later.

FIG. 3 shows the influence of the concentration of Genaminox® on the droplet size of the emulsion: 1.0% Genaminox® (dotted curve) 1.92 μm (d_{(0.1)} 1.10 μm, d_{(0.5)} 1.84 μm, d_{(0.9)} 2.90 μm); 0.5% Genaminox® (solid curve) 1.22 μm and 4.52 μm (d_{(0.1)} 0.92 μm, d_{(0.5)} 2.83 μm, d_{(0.9)} 6.11 μm). No significant changes were observed when the droplet size measurements were repeated under comparable conditions 24 hours later.

In Comparative Example 3, a solution of Genaminox® (0.5 g) in a perfume oil (HAPPY FEELING, 6 g) was dispersed at 700 rpm using a blade stirrer in an aqueous solution of Keltrol® RD (1.0 g in 91.9 g of water) and Parmetol® N 20 (0.1 g) for 15 minutes. Once the stirrer had been removed, it only took four hours for the emulsion to separate into two phases.

An emulsion can best be stabilised by a combination of surfactants which exhibits an appropriately adapted HLB value. In order to determine the HLB value which would be most favourable for a surfactant in terms of stabilising the system of the hydrophobic phase and water, the stability of several test emulsions was investigated.

Accordingly, standard solutions of Span 60 (HLB 4.7, 40 mg/ml) and Tween 60 (HLB 14.9, 40 mg/ml) in a perfume oil (HAPPY FEELING) were prepared. A mixture of varying ratios of these two surfactants (100 μl, Span/Tween 100:0, 87:13, 68:32, 50:50, 45:55, 40:60, 35:65, 30:70, 25:75, 20:80, 15:85, 10:90, 5:95, 0:100) was added to the perfume oil (900 μl) in a test vial. Water (3.80 ml) was added, and the mixture was dispersed using a vortex mixer. The stability of the prepared test emulsions was then compared in order to determine the most favourable composition (Span 60/Tween 60, 25:75) and therefore the most appropriate HLB value (12.4). Other combinations of surfactants were subjected to similar testing in accordance with the HLB optimum. The most stable compositions are compiled in FIG. 4.

FIG. 4 shows a compilation of the most favourable surfactant compositions and their corresponding ratios. The bars are centred at HLB 12.4, which has been established as the best HLB value for stabilising the system (FIG. 4). The surfactants investigated comprise Span 60 (non-ionic, HLB 4.7), Span 20 (non-ionic, HLB 8.6), Cetrimonium Chloride (cationic, HLB 10), Rewoquat (cationic, HLB 10), Trideceth-9 (non-ionic, HLB 13), Tween 60 (non-ionic, HLB 14.9), Tween 20 (non-ionic, HLB 16.7), sodium lauryl sulphate (anionic, SLS, HLB 40).

The concentration of surfactant in a scent lotion is its most important characteristic as compared to conventional solubilised oil solutions (see Comparative Example 4).

In Comparative Example 4, a solution of a solubilizer (mixture of surfactants 50% PEG-40 hydrogenated castor oil (90%), 45% Trideceth-9 and 5% propylene glycol) (10.6 g) in a perfume oil (HAPPY FEELING, 6 g) was dispersed at 2000 rpm using a blade stirrer in an aqueous solution of Parmetol® N 20 (100 mg in 83.3 g of water) for 15 minutes, but after only five minutes, the emulsion started to become increasingly transparent. A particle size determination, using a Zetasizer, confirmed that a mini-emulsion or nano-emulsion had developed.

One criterion defining a scent lotion is its low surfactant concentration, which significantly increases the droplet size of the emulsion (FIGS. 5 to 7). As the active transfer of the hydrophobic active substance depends on the droplet size (section 5 and 6) and the droplet size in turn depends on the surfactant concentration, the surfactant concentration is adjusted to such an amount necessary to formulate a stable emulsion.

FIG. 5 shows the influence of the concentration of Tween 20 on the droplet size of the emulsion: 1.0% Tween 20 (grey curve) 1.92 μm (d_{(0.1)} 1.12 μm, d_{(0.5)} 1.84 μm, d_{(0.9)} 2.86 μm); 0.5% Tween 20 (dotted curve) 2.40 μm (d_{(0.1)} 1.11 μm, d_{(0.5)} 2.21 μm, d_{(0.9)} 3.78 μm). No significant changes were observed when the droplet size measurements were repeated under comparable conditions 24 hours later.

FIG. 6 shows the influence of the concentration of surfactant on the droplet size of the emulsion; the stirring speed was 2000 rpm for all samples. The surfactant was a solubilizer consisting of 50% PEG-40 hydrogenated castor oil (90%), 45% Trideceth-9 and 5% propylene glycol. This solubilizer, applied at varying concentrations, led to the following results: 1.0% (grey curve), droplet size 2.78 μm (d_{(0.1)} 0.98 μm, d_{(0.5)} 2.25 μm, d_{(0.9)} 4.19 μm); 0.5% (black curve), droplet size 2.40 μm (d_{(0.1)} 0.99 μm, d_{(0.5)} 2.14 μm, d_{(0.9)} 4.02 μm); 0.3% (dot-dashed curve), droplet size 3.34 μm (d_{(0.1)} 0.94 μm, d_{(0.5)} 2.40 μm, d_{(0.9)} 4.90 μm); 0.2% (short-dashed curve), droplet size 4.08 μm (d_{(0.1)} 0.91 μm, d_{(0.5)} 2.73 μm, d_{(0.9)} 5.82 μm); 0.1% (long-dashed curve), droplet size 4.52 μm (d_{(0.1)} 0.93 μm, d_{(0.5)} 3.10 μm, d_{(0.9)} 6.76 μm). No significant changes were observed when the droplet size measurements were repeated under comparable conditions 24 hours later.

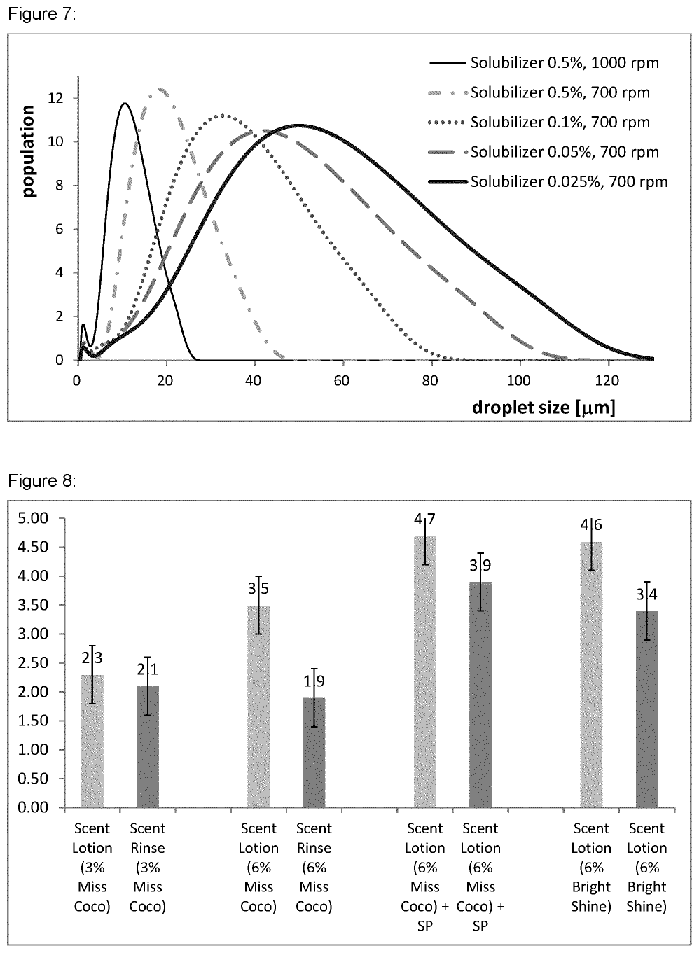

FIG. 7 shows the increase in droplet size under the influence of the concentration of surfactant (FIG. 3) and simultaneously the stirring speed (FIG. 1): 0.5% of solubilizer, dispersed at 1000 rpm (grey curve), droplet size 1.13 μm and 10.65 μm (d_{(0.1)} 1.55 μm, d_{(0.5)} 9.22 μm, d_{(0.9)} 16.51 μm); 0.5% of solubilizer, dispersed at 700 rpm (dot-dashed curve), droplet size 1.16 μm, 18.09 μm (d_{(0.1)} 6.65 μm, d_{(0.5)} 16.62 μm, d_{(0.9)} 29.13 μm); 0.1% of solubilizer, dispersed at 700 rpm (dotted curve), droplet size 1.07 μm, 32.32 μm (d_{(0.1)} 6.62 μm, d_{(0.5)} 28.12 μm, d_{(0.9)} 51.62 μm); 0.05% of solubilizer, dispersed at 700 rpm (dashed curve), droplet size 1.13 μm, 41.60 μm (d_{(0.1)} 10.59 μm, d_{(0.5)} 35.35 μm, d_{(0.9)} 67.37 μm); 0.025% of solubilizer, dispersed at 700 rpm (black curve), droplet size 1.16 μm, 49.64 μm (d_{(0.1)} 11.34 μm, d_{(0.5)} 42.24 μm, d_{(0.9)} 79.35 μm). No significant changes were observed when the droplet size measurements were repeated under comparable conditions 24 hours later.

5. Sensory Test

A test to ascertain performance after one day was conducted using green terry towels (handtowels of the firm “Brandi”) consisting of a mixture of cotton and polyester. Each panellist received their own terry towels for evaluation. The terry towels were washed, within 2 kg of ballast laundry, together with 18 g of each scent booster (scent rinse and scent lotion) containing 6% of the perfume oil GREEN LOVE. In accordance with the washing instructions, the ballast laundry and terry towels were placed in the washing machine together with the softener.

A “rinsing/starching” programme for the softener samples was started. Afterwards, the terry towels were line-dried overnight at room temperature.

Ten internal panellists conducted a blind evaluation of the scent intensity on the pieces of cloth, using a scale of 1 (odourless) to 9 (very strong).

All intensities were evaluated at the same time on dry laundry one day after washing, and the effect of rubbing the clothes was included in the test. Samples were presented in a random order. Panellists rated the intensity of each of the samples on a scale from 1 (odourless) to 9 (very strong), after which average intensities were calculated. The intensity ratings of the scent rinse and the scent lotion according to the present invention are shown in FIG. 9. For the rankings samples were presented in a random order and panellists ranked them according to a specific attribute (e.g. intensity) from 1 (weakest) to 6 (strongest). The results were evaluated statistically by a Friedmann test for overall difference between the samples, followed by paired comparison to identify significant differences among individual samples, in accordance with Praxishandbuch Sensorik (“Handbook of Best Practice in Sensor Technology”) by M Busch-Stockfisch. The rankings of the scent rinse and the scent lotion according to the present invention are also shown in FIG. 9.

The results of the sensory evaluation are shown in FIGS. 8 and 9. In FIG. 8, SP stands for solid detergent powder.

In all the sensory tests, the scent lotion was perceived to be more intense than the scent rinse (perfume solution). Remarkably, mechanical stress or heat seemed to increase the release of volatiles from the clothes treated with the emulsion.

6. Analytical Quantification of Adhered Perfume

The amount of perfume oil transferred to a piece of cloth by a washing process using a scent lotion was determined by extraction via simultaneous distillation extraction (SDE) and quantification via gas chromatography mass spectrometry (GCMS).

A selection of standardized pieces of cloth (2 kg) were washed in a “rinsing/starching” programme with 12 litres of water in a homecare washing machine (a Miele front loader), applying the scent lotion being tested as the fabric softener. One wet piece of cloth (44 g) was instantly extracted over four hours in an SDE apparatus using Et₂O/H₂O (1:4, 1250 ml) after adding NaCl (150 g) and a standard solution of diphenyl oxide (0.2 mg) in Et₂O (0.2 ml) for emulsions with d<3 μm, and of diphenyloxide (1.0 mg) in Et₂O (1.0 ml) for emulsions with d>3 μm. Simultaneously, a standard solution of scent rinse (perfume solution) or scent lotion according to the prior art (150 mg) in water (100 ml) with NaCl (20 g) and a solution of diphenyl oxide (1.000 mg) in Et₂O (1.000 ml) was extracted via SDE, also over four hours. The obtained solutions of perfume oil in Et₂O were dried overnight with anhydrous Na₂SO₄ at 4° C., filtered and concentrated up to 50 ml. The resulting extracts were quantified via GC/MS and analysed, focusing on the characteristic of SIM mass for each raw material. Integrating the SIM masses allowed the compounds to be identified and their quantities averaged and extrapolated to the whole laundry. The partial perfume oil transfer of the scent lotion was compared to that of the scent rinse (FIG. 10), and the total amount of perfume oil transferred was correlated with the droplet size of the scent lotion (FIG. 11).

FIG. 10 shows the quantitative determination of perfume oil (HAPPY FEELING) adsorption on the clothes via SDE and GC of the scent lotion according to the present invention and a conventional scent rinse. A statistical evaluation classified the data as “very significant”.

FIG. 11 shows the total amount of perfume oil adhering to a piece of cloth after a washing procedure using a Scent Lotion containing 8% perfume oil. The pieces of cloth under investigation were extracted by SDE and quantified by GC/MS. Droplet sizes are assigned in FIG. 11. The droplet size of the scent rinse was determined using a Zetasizer.

7. Preservation

Preservatives are rarely tolerated by a costumer and never healthy. Surprisingly, in a macroemulsion according to the present invention, preservatives can advantageously be substituted by a combination of antioxidants and solubilizers. To ensure, that the perfume oil does not contribute to the preserving effects, the test formulation was prepared from IPM/VOT, instead of fragrance.

TABLE 2
Perfume-free test formulation

| | | | | |
|---|---|---|---|---|
| 1 | 115262 | WATER | Water | 92.7 |
| 2 | 164419 | Isopropyl- | Isopropyl myristate | 2.5 |
| | | myristat (IPM) | | |
| 3 | 192060 | VOT | Vegetable oil triglycerides | 2.5 |
| 4 | 105433 | Vivapur MCG | Microcrystalline cellulose and | 1 |
| | | 811 F | cellulose gum (carboxymethyl | |
| | | | cellulose) | |
| 5 | 660352 | Solubilizer | PEG-40 hydrogenated castor oil | 0.5 |
| | | | and Trideceth 9 and propylene | |
| | | | glycol and water | |
| 6 | 108580 | SymDiol ® 68 | DL-Hexane-1,2-diol (CAS 6920- | 0.4 |
| | | | 22-5) | |
| 7 | 979940 | SymSave ® H | 4-Hydroxyacetophenon (CAS 99- | 0.4 |
| | | | 93-4) | |

TABLE 3
Colony forming units/g test formulation

| | | | | | | Asper- |
|---|---|---|---|---|---|---|
| | | Staphylo- | Pseudo- | | | gillus |
| | | coccus | monas | Escherichi | Candida | brasili- |
| | | aureus | aeruginosa | coli | albicans | ensis |
| | | ATCC | ATCC | ATCC | ATCC | ATCC |
| | | 6538 | 9027 | 8739 | 10231 | 16404 |
| Initial | | 470000 | 390000 | 530000 | 420000 | 590000 |
| TVC | | | | | | |
| 0 | min | 150000 | 100000 | 220000 | 150000 | 200000 |
| 2 | days | <10 | <10 | <10 | 20000 | 10000 |
| 7 | days | <10 | <10 | <10 | <10 | <10 |
| 14 | days | <10 | <10 | <10 | <10 | <10 |

The validations met the requirements of the EuPharm 2017: 5.1.3. The sample was germfree before inoculation. The sample was monitored for 14 days.

After seven days, all five test germs have been successfully eliminated by the combination of the solubilizer and the antioxidant. The results are shown in FIG. 12.

8. Update of Formulation

Similar characteristics were observed in a formulation, prepared by successive addition of the ingredients and a formulation, where perfume oil, antioxidant and surfactant had been premixed. The stirring time of the emulsion only has a low influence on the droplet size. Comparable results were obtained after a stirring time of 5 min and 2 h.

TABLE 4
Scent Lotion SPRING BREAK without preservative

| | | | | |
|---|---|---|---|---|
| 1 | 115262 | WATER | Water | 92.7 |
| 2 | 327886 | SPRING | Fragrance | 5 |
| | | BREAK EUH | | |
| 3 | 105433 | Vivapur MCG | Microcrystalline cellulose and | 1 |
| | | 811 F | cellulose gum (carboxymethyl | |
| | | | cellulose) | |
| 4 | 660352 | Solubilizer | PEG-40 hydrogenated castor oil | 0.5 |
| | | | and Trideceth 9 and propylene | |
| | | | glycol and water | |
| 5 | 108580 | SymDiol ® 68 | DL-Hexane-1,2-diol (CAS 6920- | 0.4 |
| | | | 22-5) | |
| 6 | 979940 | SymSave ® H | 4-Hydroxyacetophenon (CAS 99- | 0.4 |
| | | | 93-4) | |

9. Scale-Up to 100 kg

Water was placed in the reactor (good blending is crucial) and stirred (i.e. with Ystral X200) at room temperature at 400 rpm. The stirring speed was increased to 850 rpm and thickener Vivapur was added slowly and portion-wise to the vigorously stirring solution. The stirring of the mixture was kept at that speed for 30 minutes and the thickener was allowed to swell properly. After complete swelling, SymDiol® 68 and SymSave® H were added. The mixture for was homogenized 10 minutes at 1200-2000 rpm. The solubilizer and the perfume oil were mixed in a separate bottle. The stirring speed of the thickener dispersion was reduced to 500 rpm. The mixture of perfume oil and solubilizer was added. Stirring of the emulsion at the same rate was kept for about 10 min.

Result droplets: d₁₀ 3.41, d₅₀ 7.69, d₉₀ 15.22.

Approach with oil-concentrate:

Water was placed in the reactor (good blending is crucial) and stirred (i.e. with Ystral X200) at room temperature at 400 rpm. The stirring speed was increased to 700 to 850 rpm and thickener Vivapur was slowly and portion-wise to the vigorously stirring solution. The stirring of the mixture was kept at that speed for 20 to 30 minutes and the thickener was allowed to swell properly. After complete swelling, the mixture was homogenized for 10 minutes at 1200-2000 rpm. The solubilizer, SymDiol® 68 and SymSave® H were mixed with the perfume oil in a separate bottle. The stirring speed of the thickener dispersion was reduced to 300 rpm or less. The mixture of perfume oil and solubilizer, SymDiol® 68 and SymSave® H was added. The stirring of the emulsion at the same rate was kept for 5 to 10 min.
Result droplets: d₁₀ 2.7, d₅₀ 6.5, d₉₀ 14.1.

10. Droplet Charge in Dependence of the Emulsifier System

The sheer force (stirring speed) is the main influence on the droplet size. However, the droplet size range, induced by the sheer force depends on the character of the emulsifier system. Therefore, five different emulsifier systems (non-ionic, cationic, anionic) have been evaluated.

All samples were prepared on a 50 g scale in the Speedmixer. The formulations contained Vivapur MCG 811 F (microcrystalline cellulose and cellulose gum, 1.00%), perfume oil (Happy Feeling, 5.00%), surfactant (different mixtures, 0.50%) and preservative (Parmetol N20=Bronopol, 2-Octyl-2H-isothiazol-3-on, 0.1%).

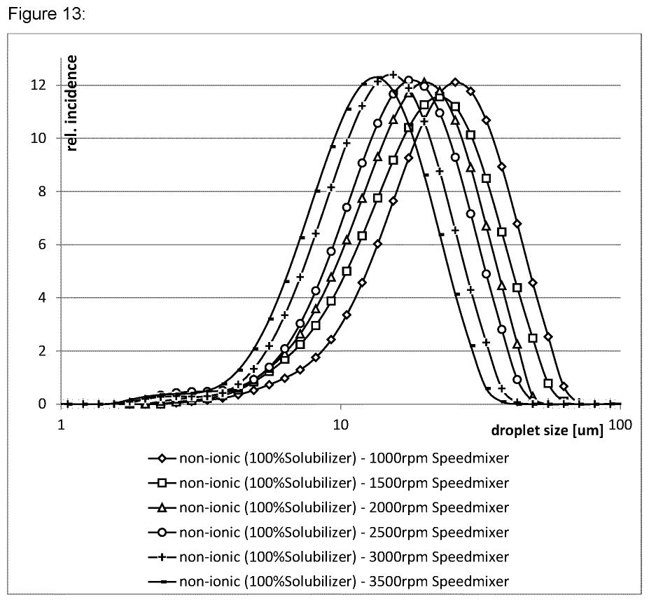

FIG. 13 shows the droplet size distribution of the Scent Lotion formulation prepared on a 50 g scale with Vivapur MCG 811 F (1.00%), perfume oil (Happy Feeling, 5.00%), non-ionic solubilizer (PEG-40 hydrogenated castor oil, Trideceth 9 and DPG, 0.50%) and preservative (Parmetol N20, 0.1%) in the Speedmixer. The sheer stress was varied (1000 to 3500 rpm). Droplet sizes were measured via Mastersizer (Table 5).

TABLE 5
Droplet sizes of the Scent Lotion with non-ionic
surfactant in dependence of the stirring speed

| | Non-ionic (100% Solubilizer) | | | | | |
|---|---|---|---|---|---|---|
| | 1000 | 1500 | 2000 | 2500 | 3000 | 3500 |
| | rpm | rpm | rpm | rpm | rpm | rpm |
| Dx (10) | 11.244 | 9.208 | 8.612 | 7.999 | 7.21 | 6.192 |
| Dx (50) | 23.614 | 20.245 | 18.119 | 16.554 | 14.153 | 12.437 |
| Dx (90) | 42.015 | 36.97 | 32.031 | 29.454 | 25.076 | 22.25 |

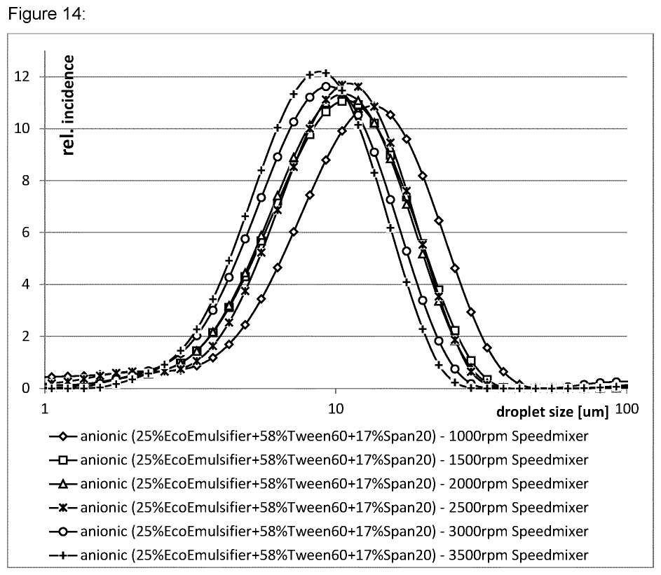

FIG. 14 shows the droplet size distribution of the Scent Lotion formulation, prepared on a 50 g scale with Vivapur MCG 811 F (microcrystalline cellulose and cellulose gum, 1.00%), perfume oil (Happy Feeling, 5.00%), anionic surfactant mixture (25% EcoEmulsifier+58% Tween60+17% Span20, 0.50%) and preservative (Parmetol N20=Bronopol, 2-Octyl-2H-isothiazol-3-on, 0.1%) in the Speedmixer. The sheer stress was varied (1000 to 3500 rpm). Droplet sizes were measured via Mastersizer (Table 6).

TABLE 6
Droplet sizes of the Scent Lotion with anionic
surfactant in dependence of the stirring speed

| | Anionic surfactant mixture | | | | | |
|---|---|---|---|---|---|---|
| | (25% EcoEmulsifier + 58% Tween60 + 17% Span20) | | | | | |
| | 1000 | 1500 | 2000 | 2500 | 3000 | 3500 |
| | rpm | rpm | rpm | rpm | rpm | rpm |
| Dx (10) | 5.088 | 4.725 | 4.799 | 4.901 | 4.416 | 4.347 |
| Dx (50) | 12.211 | 10.182 | 10.048 | 10.338 | 8.979 | 8.437 |
| Dx (90) | 23.466 | 19.553 | 19.08 | 19.048 | 16.823 | 15.4 |

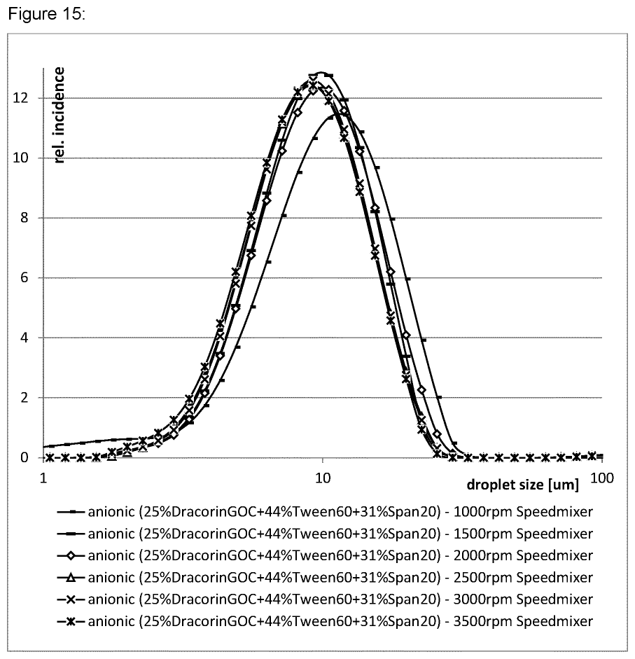

FIG. 15 shows the droplet size distribution of the Scent Lotion formulation, prepared on a 50 g scale with Vivapur MCG 811 F (1.00%), perfume oil (Happy Feeling, 5.00%), anionic surfactant mixture (25% Dracorin GOC=Glyceryl Oleate Citrate, Caprylic/Capric Triglyceride [2]+44% Tween60+31% Span20, 0.50%) and preservative (Parmetol N20, 0.1%) in the Speedmixer. The sheer stress was varied (1000 to 3500 rpm). Droplet sizes were measured via Mastersizer (Table 7).

TABLE 7
Droplet sizes of the Scent Lotion with anionic
surfactant in dependence of the stirring speed

| | Anionic surfactant mixture | | | | | |
|---|---|---|---|---|---|---|
| | (25% DracorinGOC + 44% Tween60 + 31%Span20) | | | | | |
| | 1000 | 1500 | 2000 | 2500 | 3000 | 3500 |
| | rpm | rpm | rpm | rpm | rpm | rpm |
| Dx (10) | 4.69 | 4.949 | 4.99 | 4.729 | 4.754 | 4.507 |
| Dx (50) | 10.438 | 9.354 | 9.539 | 8.919 | 8.934 | 8.645 |
| Dx (90) | 19.292 | 16.235 | 17.185 | 16.202 | 16.08 | 15.46 |

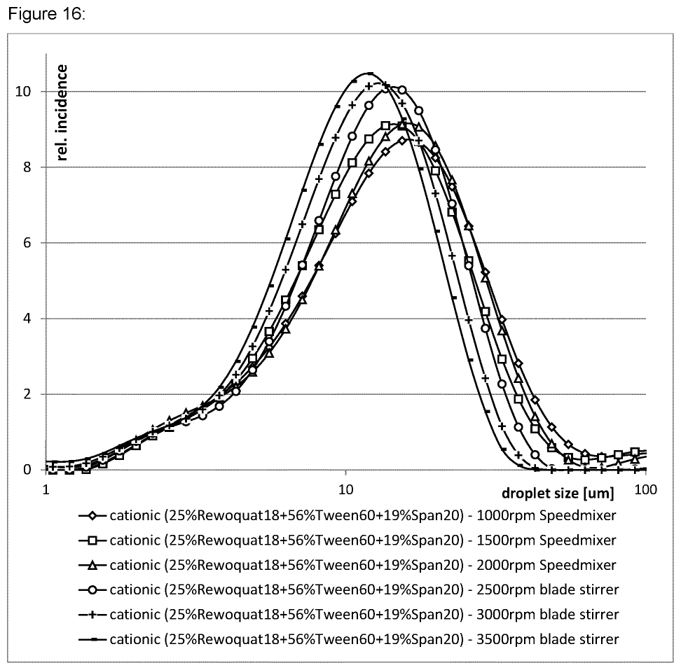

FIG. 16 shows the droplet size distribution of the Scent Lotion formulation, prepared on a 50 g scale with Vivapur MCG 811 F (1.00%), perfume oil (Happy Feeling, 5.00%), cationic surfactant mixture (25% RewoquatWE18=Dihydrogenated Tallowethyl Hydroxyethylmonium Methosulfate CAS 91995-81-2, 67-63-0+56% Tween60+19% Span20, 0.50%) and preservative (Parmetol N20, 0.1%) in the Speedmixer. The sheer stress was varied (1000 to 3500 rpm). Droplet sizes were measured via Mastersizer (Table 8).

TABLE 8
Droplet sizes of the Scent Lotion with cationic
surfactant in dependence of the stirring speed

| | Cationic surfactant mixture | | | | | |
|---|---|---|---|---|---|---|
| | (25% Rewoquat18 + 56% Tween60 + 19% Span20) | | | | | |
| | 1000 | 1500 | 2000 | 2500 | 3000 | 3500 |
| | rpm | rpm | rpm | rpm | rpm | rpm |
| Dx (10) | 4.722 | 4.902 | 4.451 | 5.011 | 4.483 | 4.157 |
| Dx (50) | 14.274 | 13.174 | 13.952 | 12.919 | 11.503 | 10.542 |
| Dx (90) | 33.391 | 30.165 | 30.623 | 26.403 | 23.144 | 20.669 |

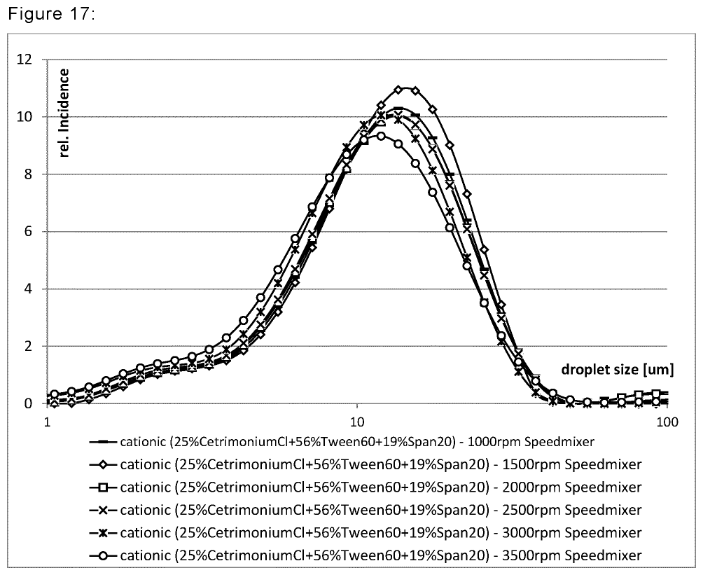

FIG. 17 shows the droplet size distribution of the Scent Lotion formulation, prepared on a 50 g scale with Vivapur MCG 811 F (1.00%), perfume oil (Happy Feeling, 5.00%), cationic surfactant mixture (25% CetrimoniumCl+56% Tween60+19% Span20, 0.50%) and preservative (Parmetol N20, 0.1%) in the Speedmixer. The sheer stress was varied (1000 to 3500 rpm). Droplet sizes were measured via Mastersizer (Table 9).

TABLE 9
Droplet sizes of the Scent Lotion with cationic
surfactant in dependence of the stirring speed

| | Cationic surfactant mixture | | | | | |
|---|---|---|---|---|---|---|
| | (25% CetrimoniumCl + 56% Tween60 + 19% Span20) | | | | | |
| | 1000 | 1500 | 2000 | 2500 | 3000 | 3500 |
| | rpm | rpm | rpm | rpm | rpm | rpm |
| Dx | 4.824 | 5.101 | 4.763 | 4.555 | 3.999 | 3.602 |
| (10) | | | | | | |
| Dx | 12.509 | 12.703 | 12.388 | 12.106 | 11.114 | 10.63 |
| (50) | | | | | | |
| Dx | 25.706 | 23.737 | 26.46 | 25.174 | 22.946 | 23.44 |
| (90) | | | | | | |

The droplet size of macroemulsions formed from non-ionic solubilizer showed the most sensitivity towards the shear stress induced. All ionic surfactant combinations were much less influenced by the stirring speed. The droplet size distribution is more uniform with ionic surfactant combinations (see FIGS. 13 to 18). A formation of droplets larger than d₅₀=20 μm was not possible under standard conditions with electrostatically loaded droplets rather than with non-ionic solubilizers.

11. Adhesion of Perfume Oil in Dependence of the Droplet Charge

The influence of the surfactant properties on the perfume oil transfer to the clothes was investigated. To compare the macroemulsions, the formulations with most similar droplet sizes were chosen, applied to laundry via washing machine and the perfume oil on the clothes was quantified.

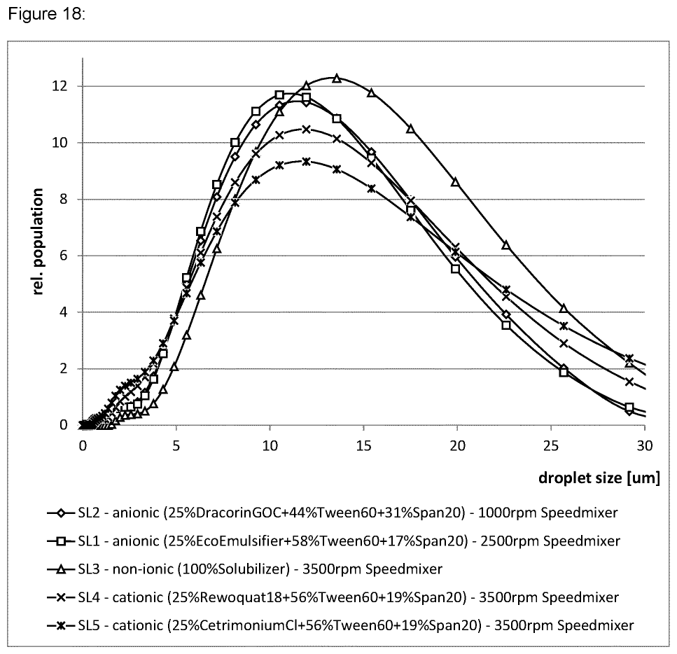

FIG. 18 represents an assembly of Scent Lotion formulations of different surfactant compositions with similar droplet sizes, adjusted by the applied sheer stress. All Scent Lotion formulations were prepared on a 50 g scale with Vivapur MCG 811 F (microcrystalline cellulose and cellulose gum, 1.00%), perfume oil (Happy Feeling, 5.00%), surfactant mixture (0.50%) and preservative (Parmetol N20, 0.1%) in the Speedmixer (Table 10).

TABLE 10
Droplet sizes of the Scent Lotions, prepared for comparison of their stability and perfume oil transfer on laundry

| | 2 | | | 4 | |
|---|---|---|---|---|---|
| | Anionic surfactant | 1 | 3 | Cationic surfactant | 5 |
| | mixture (25% | Anionic surfactant | Non-ionic (100% | mixture (25% | Cationic surfactant |
| | Dracorin GOC | mixture (25% | Solubilizer = PEG- | RewoquatWE 18 (TEA- | mixture (25% |
| | [2] + 44% | EcoEmulsifier [1] + | 40 hydrogenated | Esterquat-methosulfat) + | CetrimoniumCl + |
| | Tween60 + 31% | 58% Tween60 + | castor oil, Trideceth- | 56% Tween60 + | 56% Tween60 + |
| | Span20) | 17% Span20) | 9, Propylenglycol) | 19% Span20) | 19% Span20) |
| | 1000 rpm | 2500 rpm | 3500 rpm | 3500 rpm | 3500 rpm |
| Dx (10) | 4.69 | 4.901 | 6.192 | 4.157 | 3.602 |
| Dx (50) | 10.438 | 10.338 | 12.437 | 10.542 | 10.63 |
| Dx (90) | 19.292 | 19.048 | 22.25 | 20.669 | 23.44 |

Quantification of perfume oil transfer from the laundry perfume formulation to the laundry by the washing procedure. Pieces of cloth were washed with the Scent Lotion formulations and extracted via SDE. The extracted perfume oil was quantified via GC and extrapolated to the complete clothes in the washing machine.

TABLE 11
Experimental procedure of the analytical approach to determine
the effect of different surfactants on the perfume oil transfer
to the clothes in the washing/laundry process

| | |
|---|---|
| Washing procedure: | Program Starching (“Stärken”); 18 g base with 8% |
| | perfume oil washed with Starching (“Stärken”) in |
| | Miele Frontloader. |
| Fabricload: | 4 Towels Cotton/Frottee, plus Dummyload (together |
| | ~2 kg). |
| Extraction | 1 Towel with 150 g NaCl and 200 μg internal Std. |
| | Diphenyl Oxide and 1000 mL dest. Water, |
| | extracting 4 hours via SDE. |
| Preperation of Fresh | 0.15 g base with 5% perfume oil 100 ml water, |
| washing liquid for | extracted with 30 g NaCl and 500 μg internal Std. |
| analysis: | Diphenyl Oxide, extracting 4 hours via SDE. |
| Measurement: | The extracts are concentrated (towel extract) to |
| | 10 mL, the washing liquid extracts are used directly, |
| | then measured liquid in splitless mode. The |
| | quantification is performed via GC-MS using one |
| | characteristic SIM mass for each raw material. |

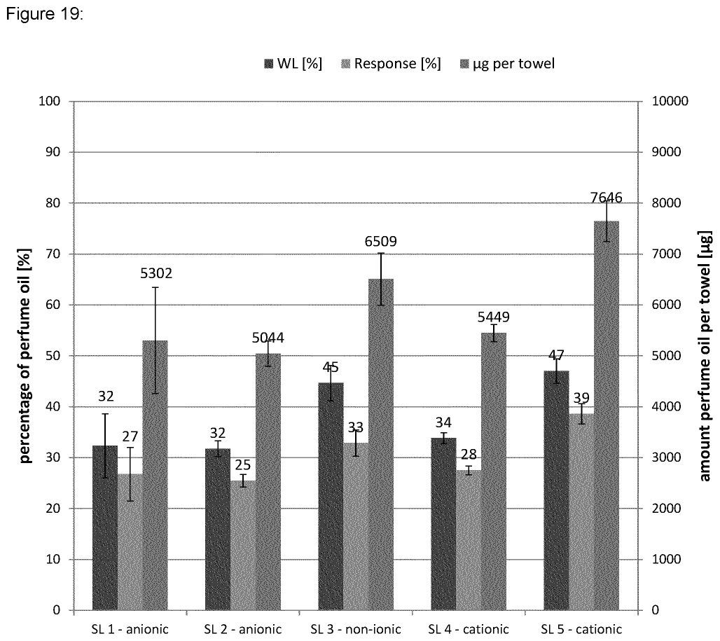

FIG. 19 shows the quantification of perfume oil transfer to fabric.
SL 1—anionic: Surfactant was 25% EcoEmulsifier+58% Tween60+17% Span20−2500 rpm prepared in a SpeedMixer.
   ζ−56.1 mV. d₁₀ 4.90 μm, d₅₀ 10.34 μm, d₉₀ 19.05 μm.
SL 2—anionic: Surfactant was 25% DracorinGOC+44% Tween60+31% Span20−1000 rpm prepared in a SpeedMixer.
   ζ−63.6 mV. d₁₀ 4.69 μm, d₅₀ 10.44 μm, d₉₀ 19.29 μm.
SL 3—non-ionic: Surfactant was 100% Solubilizer (PEG-40 hydrogenated castor oil, Trideceth-9, propylene glycol, water)−3500 rpm prepared in a SpeedMixer.
   ζ−11.7 mV. d₁₀ 6.19 μm, d₅₀ 12.44 μm, d₉₀ 22.25 μm.
SL 4—cationic: Surfactant was 25% RewoquatWE18 (TEA-Esterquat-methosulfat)+56% Tween60+19% Span20−3500 rpm prepared in a SpeedMixer.
   ζ−42.0 mV. d₁₀ 4.16 μm, d₅₀ 10.54 μm, d₉₀ 20.67 μm.
SL 5—cationic: Surfactant was 25% Cetrimoniumchlorid+56% Tween60+19% Span20−3500 rpm prepared in a SpeedMixer.
   ζ−46.0 mV. d₁₀ 3.60 μm, d₅₀ 10.63 μm, d₉₀ 23.44 μm.

The negative ζ potential of the macroemulsions made from cationic surfactant mixtures at pH 7.0 indicates an interaction between the positively charged droplets and the anionic thickener carboxymethyl cellulose. Considering the thickener layer on the surface of the droplets, their size can be estimated a little smaller than measured via mastersizer.

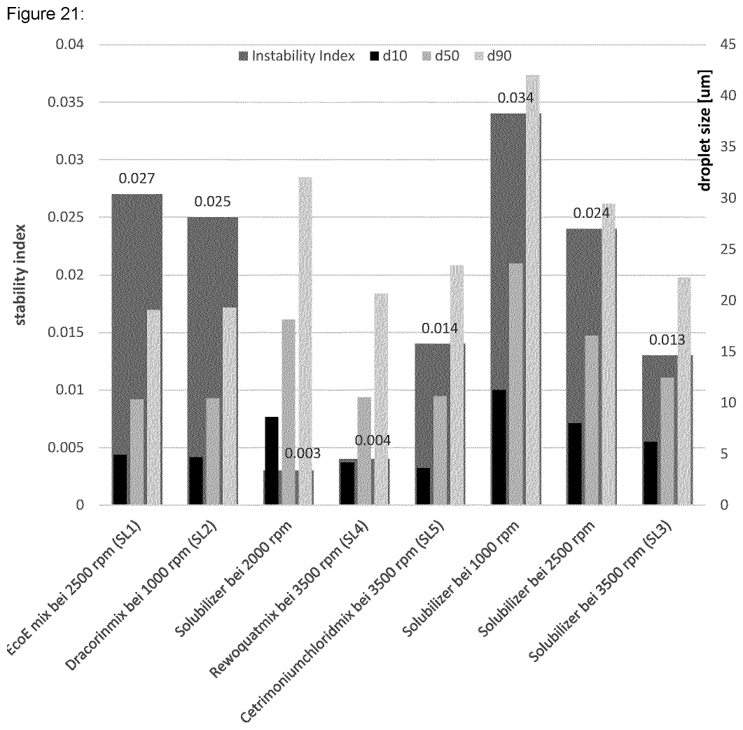

At first glance, no clear correlation can be observed between droplet charge and fragrance transfer, though the anionic surfactant stabilized emulsions appear less effective and the best perfume oil transfer was observed in cationic SL 5. However, the stability of the emulsion can be expected to affect the perfume oil transfer, as well, and has to be considered. The samples with cationic droplets (SL4 and SL5) appear to be more stable than those with anionic droplets (SL1 and SL2) (see FIG. 21).

12. Shelf Life Stability

Up to date, samples of the scent lotion with random perfume oils have been stored at room temperature on shelf. A separation of the phases has never been observed at droplet sizes smaller than d=30 μm. Therefore, formulations with different surfactant properties and droplet sizes have been investigated by Lumisizer and Rheology.

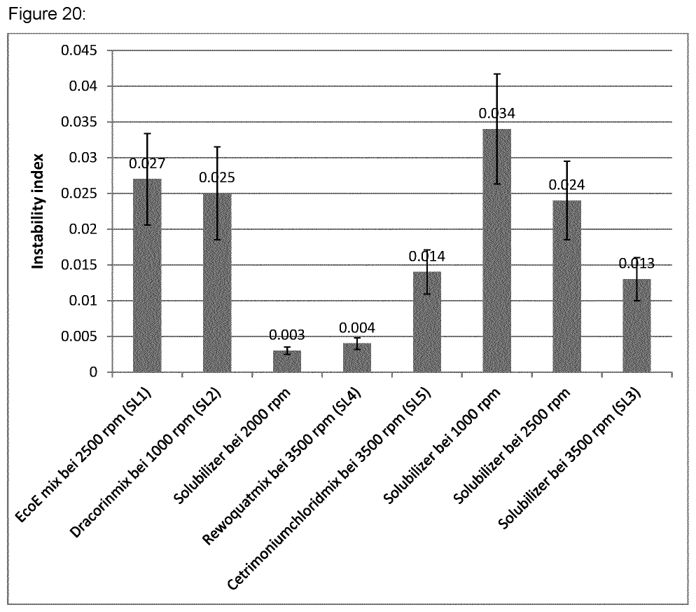

FIG. 20 represents samples of the scent lotions prepared from different surfactant compositions at different stirring speeds have been stressed at 3000 rpm and 40° C. over 1.75 h in a Lumisizer. Their instability index was determined by evaluation of the creaming, as indicated by time-dependent transmission. From left to right: SL1, SL2, SL3, SL4, SL5, shear stress dependent instability index.

The instability factors/instability indices were determined on a Lumisizer (6102-41) in a 2 mm cuvette (LUM 2 mm, PA, Rectangular Synthetic Cell 110-134××, 2×8×22 mm). All samples were measured simultaneously over 1.75 h at 40° C. and 3000 rpm. Detection was at 865 nm (light factor 0.25-6.00). All curves were normalized, dynamically baseline corrected and moving averaged.

The observed instability indices have been compared to the droplet size of the emulsions under investigation.

TABLE 12
Compilation of instability indices (measured via Lumisizer) and
the corresponding droplet sizes (measured by Mastersizer).

| | | Stan- | | | |
|---|---|---|---|---|---|
| | Instabil- | dard | | | |
| | ity | devia- | | | |
| Surfactant | Index | tion | d10 | d50 | d90 |
| SL1 EcoE mix at 2500 | 0.027 | 0.0064 | 4.901 | 10.338 | 19.048 |
| rpm | | | | | |
| SL2 Dracorinmix at 1000 | 0.025 | 0.0065 | 4.69 | 10.438 | 19.292 |
| rpm | | | | | |
| SL3 Solubilizer at 2000 | 0.003 | 0.0005 | 8.612 | 18.119 | 32.031 |
| rpm | | | | | |
| SL4 Rewoquatmix at | 0.004 | 0.0008 | 4.157 | 10.542 | 20.669 |
| 3500 rpm | | | | | |
| SL5 Cetrimonium- | 0.014 | 0.0031 | 3.602 | 10.63 | 23.44 |
| chloridmix at 3500 rpm | | | | | |
| Solubilizer at 1000 rpm | 0.034 | 0.0077 | 11.244 | 23.614 | 42.015 |
| Solubilizer at 2500 rpm | 0.024 | 0.0055 | 7.999 | 16.554 | 29.454 |
| Solubilizer at 3500 rpm | 0.013 | 0.003 | 6.192 | 12.437 | 22.25 |

FIG. 21 is a visualization of the correlation between droplet size and instability index. The surfactant compositions are:
SL1—anionic: EcoE mix=25% EcoEmulsifier [1]+58% Tween60+17% Span20, anionic.
SL2—anionic: Dracorinmix=25% DracorinGOC [2]+44% Tween60+31% Span20, anionic.
SL3—non-ionic: Solubilizer=PEG-40 hydrogenated Castor oil, Trideceth-9, Propyleneglycol, Water, non-ionic.
SL4—cationic: Rewoquatmix=25% RewoquatWE18 (TEA-Esterquat-methosulfat)+56% Tween60+19% Span20, cationic.
SL5—cationic: Cetrimoniumchloridmix=25% Cetrimoniumchlorid+56% Tween60+19% Span20, cationic.

The lower the instability index, the more stable the macroemulsion sample is. The trend in the samples prepared from the solubilizer indicates that the instability index increases in dependence of the droplet size. The samples with cationic droplets (SL4 and SL5) appear to be more stable than those with anionic droplets (SL1 and SL2). Possibly, the anionic thickener stabilizes the surface of the cationic droplets against coalescence.

Some hints on the stability of the macroemulsions were derived from rheological measurements, using a RheoStress RS1, P60 Ti L parallel geometry. An oscillation measurement was run at a linear temperature ramp (5 to 45° C. over 800 s) at a frequency of f=0.2000 to 1.000 Hz (linear range of oscillation ramp has been predetermined). The samples were submitted to five temperature cycles from 5 to 45° C. and back to 5° C. The rheological analysis were performed in oscillatory flow conditions using a rotational rheometer, Rheostress RS1, equipped with a P60 Ti L in a parallel geometry (fixed gap 1,000 mm). Before carrying out the rheological analysis, all samples were stored at room temperature for at least 2 days before measurement. The temperature sweep analysis was performed using a Peltier system by varying the temperature from 5 to 45° C. (5 cycles, linear temperature gradient of 3 K/minΔ0.05 K) at a frequency of 0.2-1.0 Hz) in order to investigate the dependence of the viscoelastic properties (G′, G″) of different samples on temperature.

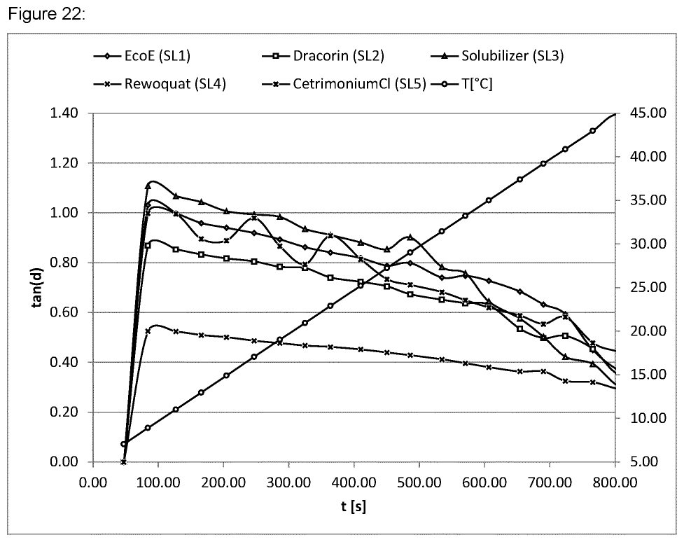

FIG. 22 represents a combination of elasticity and loss modulus: tan(d)=G′/G″. Influence of the temperature (5 to 45° C.) on five samples with different surfactant compositions.

The development of G′ and G″ is an indicator for the temperature-stability of a sample. The bigger the influence of temperature, the lower the shelf live. In the first heating cycle, the development of G″ is comparatively constant, while G′ increases strongly at elevated temperature. The sharp increasing of G′ at T>40° C. indicates a sensitivity of all investigated macroemulsions against elevated temperatures. The sample stabilized with Rewoquat appears to be most stable, as already indicated by the stability index via Lumisizer, while the non-ionic solubilizer yields the most temperature-sensitive samples. The correlation between temperature dependence of G′ and ζ-potential indicates, that the electronic stabilization of the droplets is of some importance.

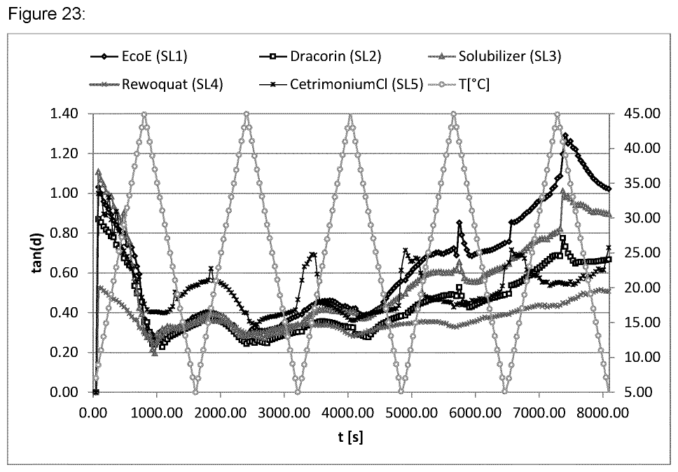

FIG. 23 represents a combination of elasticity and loss modulus: tan(d)=G′/G″. Influence of the temperature (5.00 to 45.00° C.) on five samples with different surfactant compositions. Temperature dependence was monitored over 5 heating/cooling cycles.

None of the investigated samples shows a constant course of G′ over the repeated temperature cycles. The shift of the G′-courses indicates that no sample is thermodynamically stable (which of course is not to be expected from macroemulsions). The typical course of one temperature cycle, however, is maintained in the samples, prepared from cationic surfactant systems. They appear to be most stable. Probably, the cationic oil droplets are stabilized by the anionic surfactant via ionic interactions, forming a kind of thickener-layer on their surface. The curves of G″ confirm the observation of the G′ curves.

13. Concentrate

Preparation of a Scent Lotion concentrate has been evaluated in order to reduce the water content and therefore the dosage of the formulation in the laundry machine. A concentrate enables the reduction of packaging material and makes the product more sustainable.

FIG. 24 shows the relation between droplet size and perfume oil concentration. The Scent Lotion concentrate samples were prepared with Vivapur MCG 811 F (1.00%), SymDiol® 68 (0.4%), SymSafe® H (0.4%) and different concentrations of perfume oil “Happy Feeling” (HF) and different concentrations of solubilizer at 2000 rpm in the Speedmixer. The ratio between solubilizer and perfume oil were generally 1:10, with exception of the purple curve (1:23).

The droplet size decreases with rising concentration of the organic phase. The ratio solubilizer:perfume oil thus has to be adjusted in a concentrate accordingly in order to obtain appropriate droplet sizes. Favorably, the solubilizer concentration is from 1 to 20% by weight, more preferably 4 to 15% by weight and most preferably from 8 to 12% by weight, based on the total weight of the oily or dispersed phase.

14. Perfume Oil Properties

The perfume oil transfer of a laundry perfume heavily depends on the properties of the perfume oil. Therefore, the amount of different fragrance compounds was quantified on the cloth and divided by its percentage of the perfume oil.

In FIG. 25a different emulsions with the same concentration of perfume substance but with different droplet sizes were compared. The fragrance compounds are ordered by their relative quantitative transfer to the clothes in dependency on their droplet size. The bigger the difference of the relative quantitative transfer in relation to the droplet size the bigger the effect of the Scent Lotion and the better the properties of the perfume substance are.

FIG. 25b shows the log P value and the water solubility of the perfume substances listed in FIG. 25a: Light grey=water solubility as experimentally determined [mg/l]; dark grey=log P value (octanol:water) as experimentally determined.

Fragrance compounds with low water solubility and high log P value appear to adhere better to the clothes. Fragrances with a water solubility≤100 mg/I and a log P≥3.0 seem to be transferred best to the laundry and are most recommendable for application in the scent lotion.

1. An oil-in-water macroemulsion, comprising:
an aqueous phase;
an oily phase comprising at least one hydrophobic active substance;
at least one surfactant;
at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches, modified celluloses, and mixtures thereof;
wherein the amount of the surfactant is from 0.4 to 25% by weight, based on the total weight of the oily phase.

2. The oil-in-water macroemulsion according to claim 1, wherein the at least one hydrophobic active substance is selected from the group consisting of perfume substances, perfume oils, aroma substances, aromas, active skin-product ingredients, active pharmaceutical ingredients, dyes, UV-active substances, optical brighteners, bodying agents, drape and form control agents, smoothness agents, static control agents, wrinkle control agents, sanitising agents, disinfecting agents, germ control agents, mould control agents, mildew control agents, antiviral agents, antimicrobials, drying agents, stain resistance agents, soil release agents, malodour control agents, fabric freshening agents, dye fixatives, colour maintenance agents, colour restoring/rejuvenating agents, anti-fading agents, anti-abrasion agents, wear resistance agents, fabric integrity agents, anti-wear agents, rinsing aids, UV protection agents, sun fade inhibitors, insect repellents, anti-allergenic agents, flame retardants, water-proofing agents, fabric softening agents, shrinkage resistance agents, stretch resistance agents, and mixtures thereof.

3. The oil-in-water macroemulsion according to claim 1, wherein the proportion of the at least one hydrophobic active substance is 0.1 to 75% by weight, relative to the total weight of the macroemulsion composition.

4. The oil-in-water macroemulsion according to claim 1, wherein the oily phase further comprises a hydrophobic active substance in a microcapsule form.

5. The oil-in-water macroemulsion according to claim 1, wherein the at least one surfactant has an HLB value of 8 to 18, in particular an HLB value of 11 to 14.

6. The oil-in-water macroemulsion according to claim 1, wherein the at least one surfactant is selected from the group consisting of non-ionic surfactants, anionic surfactants, cationic surfactants, amphoteric surfactants and mixtures thereof.

7. The oil-in-water macroemulsion according to claim 1, wherein the at least one stabiliser is selected from the group consisting of polyacrylate thickener, xanthan gum, gellan gum, guar gum, alginic acid, alginate, agar-agar, carrageenan, welan gum, locust bean gum, tragacanth, gum arabic, pectin, polyoses, starch, dextrin, gelatine, casein, modified starch, modified cellulose and mixtures thereof.

8. The oil-in-water macroemulsion according to claim 1, wherein the average oil droplet size is >1 μm.

9. The oil-in-water macroemulsion according to claim 1, comprising:
25 to 99.9% by weight of the aqueous phase;
0.1 to 75% by weight of the oily phase comprising at least one hydrophobic active substance;
0.004 to 18% by weight of surfactant;
up to 1% by weight of stabiliser; and optionally
up to 1% by weight of additives and/or adjuvants;
based on the total weight of the macroemulsion.

10. A method for preparing an oil-in-water macroemulsion, comprising the steps of:
(1-i) providing an aqueous phase by mixing and dissolving at least one in an aqueous solution;
(1-ii) providing an oily phase by mixing and dissolving at least one hydrophobic active substance and/or at least one hydrophobic active substance in a microcapsule form, and at least one surfactant in an oily solution; and
(1-iii) dispersing the oily phase in the aqueous phase by stirring, shaking, pressing or otherwise inducing sheer forces, to obtain an oil-in-water macroemulsion;
or
(2-i) providing an aqueous phase by mixing and dissolving at least one surfactant and at least one stabiliser in an aqueous solution;
(2-ii) providing an oily phase by mixing and dissolving at least one hydrophobic active substance and/or at least one hydrophobic active substance in a microcapsule form in an oily solution; and
(2-iii) dispersing the oily phase in the aqueous phase by stirring, shaking, pressing or otherwise inducing sheer forces, to obtain a macroemulsion;
wherein the amount of the surfactant is from 0.4 to 25% by weight, based on the total weight of the oily phase.

11. The method according to claim 10, wherein the oil-in-water macroemulsion is prepared with a blade stirrer at a stirring or shaking rate of 500 to 2000 rpm, or with a Ystral stirrer at minimum stirring rate.

12. A method for using a hydrophobic active substance, comprising the steps of:
providing an oil-in-water macroemulsion according to claim 1; and
bringing the oil-in-water macroemulsion, in contact with a subject or object.

13. The method according to claim 12, wherein the subject is selected from the group consisting of skin, hair, beard or fur, or the object is selected from the group consisting of fabrics, clothes, surfaces, natural fibres, synthetic fibres, wood, food, cosmetics, pharmaceuticals and pet foods.

14. The method according to claim 12, wherein the providing and contacting are in a washing or rinsing process.

15. (canceled)

16. (canceled)

17. A food, cosmetic, personal care products, homecare product, pharmaceutical or pet food comprising an oil-in-water macroemulsion according to claim 1.
